(19) United States
(12) Patent Application Publication
(10) Pub. No.: US 2018/0290123 A1
(43) Pub. Date: Oct. 11, 2018

(54) CORONA DISCHARGE REACTOR AND METHOD FOR USING

(71) Applicants: Goran Nadezda Jovanovic, Corvallis, OR (US); Alexandre F.T. Yokochi, Corvallis, OR (US); Yu Miao, Corvallis, OR (US); Justin Pommerenck, Corvallis, OR (US); Yousef Alanazi, Corvallis, OR (US); Peter Kreider, Corvallis, OR (US)

(72) Inventors: Goran Nadezda Jovanovic, Corvallis, OR (US); Alexandre F.T. Yokochi, Corvallis, OR (US); Yu Miao, Corvallis, OR (US); Justin Pommerenck, Corvallis, OR (US); Yousef Alanazi, Corvallis, OR (US); Peter Kreider, Corvallis, OR (US)

(73) Assignee: Oregon State University, Corvallis, OR (US)

(21) Appl. No.: 15/948,776

(22) Filed: Apr. 9, 2018

Related U.S. Application Data
(60) Provisional application No. 62/483,799, filed on Apr. 10, 2017.

(51) Int. Cl. B01J 19/00; C07C 2/80

(52) U.S. Cl. CPC B01J 19/0093; C07C 2/80; B01J 2219/0093; B01J 2219/00869; B01J 2219/00822; B01J 2219/0086; B01J 2219/00853

(57) ABSTRACT

Disclosed microreactors operate in an electrical discharge mode, such as a pulse mode, an arc mode or a corona discharge mode, and most preferably in a corona discharge mode. A microreactor may comprise multiple, simultaneously operating corona discharges. The microreactor typically has at least one feature measured on a millimeter scale. Certain disclosed microreactors comprised multiple reactor plates in a stack. Each plate comprised plural corona discharge electrodes positioned in series along each of plural corresponding microchannels. A method for using disclosed microreactors and systems comprising disclosed microreactors, such as for chemical transformations, fluid purifications, or both, also is disclosed.

### CROSS REFERENCE TO RELATED APPLICATION

This application claims the benefit of the earlier filing date of U.S. Provisional Application No. 62/483,799, filed Apr. 10, 2017, which is incorporated herein by reference.

### ACKNOWLEDGMENT OF GOVERNMENT SUPPORT

This invention was made with government support under National Science Foundation (NSF) Award No. 1134249, entitled “Chemical Reaction Activation in Microreactors through Corona Discharge,” and under Department of Energy Award No. DE-AR-0000679. The United States government has certain rights in the invention.

### FIELD

This application concerns reactors capable of operating in a corona discharge mode, particularly microscale-based corona discharge reactors, arrays of such reactors, systems comprising at least one corona discharge reactor, and a method for using such reactors and systems, such as for fluid purification and/or chemical transformations.

### BACKGROUND

A practical technology is required for performing chemical transformations and/or fluid purifications on both small scales, as well as industrial scales. A preferred process would be one that could be widely deployed for distributed processing applications. One example of such a chemical transformation and/or chemical purification process is the conversion of methane to liquid fuels. There are at least three distinct processes that need to be considered: (1) converting small sources of methane that are not otherwise economically viable (a.k.a., stranded methane); (2) fueling vehicles with liquid fuels derived from natural gas produced at the refueling point; and (3) large scale industrial conversion of natural gas to ultra-low sulfur liquid fuels. The scale of the flared natural gas resource worldwide has been estimated at about 5×10¹² cu ft per year and would be worth approximately $30 billion if recovered, with 250×10⁹ cu ft (ca. 5,000 kt methane) flared in the United States.

Animal manure management, such as on a large scale dairy, provides another example of the scale of the stranded methane resource. A 2013 estimate concluded that 2,456 kt methane were generated by manure management (equivalent to 61.4 MMt CO₂e); 1,000 kt methane (25.2 MMt CO₂e) resulting from pneumatic bleeds and tank venting in the energy industries; and 5,000 kt methane flared (15 MMt CO₂). Since methane is burned to form carbon dioxide, methane production corresponds to CO₂ production, which is a significant contributor to global warming. These processes provide an opportunity to generate approximately 8,500 kt liquid fuels (about 3.2 MM gallons liquid fuels). If this production displaced an equivalent amount of conventional fuels, it would reduce CO₂ emissions by 101 MMt emissions per year. For individuals and industrial entities that desire to generate their own liquid hydrocarbons by condensation of natural gas, an efficient conversion methodology would provide a significant financial opportunity that would increase production of liquid transportation fuels and reduce energy imports, while also reducing carbon emissions. There is currently no technology that can effect this conversion for small resources in a practical manner.

There also is a substantial opportunity at larger scale conversion considerations. For example, a large scale opportunity exists to fuel some of the 250 million road vehicles in the United States, which currently consume 25% of the 100 quads that the United States uses annually. The United States transportation sector is heavily dependent on petroleum with about 93% of transportation fuels derived from petroleum in 2012. The United States uses about 13.5 million barrels petroleum per day. Natural gas use by motor vehicles is a very small fraction of the total transportation energy use. While recent low prices and abundant domestic supplies of natural gas have increased interest in its use as a motor fuel, its deployment currently is unlikely due to consumer's desire to continue to use existing gasoline or diesel vehicles, and desire to avoid conversion costs and technology compromises. However, a device and method for its use that would enable energy efficient conversion of natural gas to gasoline or diesel would substantially decrease, and almost eliminate, the United States dependence on petroleum imports. This would allow the United States to use its vast domestic resource of natural gas, while maintaining low carbon emissions. In fact, if all shale and/or tar sands oil production was replaced with natural-gas-derived liquid fuels, very significant reductions in total carbon emissions from transportation would be achieved.

### SUMMARY

The present device, system comprising the device, and method for their use, addresses the needs discussed above. Certain disclosed embodiments concern microreactors that are configured to operate in an electrical discharge mode, such as a pulse mode, an arc mode or a corona discharge mode, and most preferably are operable in a corona discharge mode. The microreactor may comprise a single emitter electrode and a plate counter electrode, and may comprise a flow-through plasma microreactor system. The microreactor may be configured to produce multiple, simultaneously operating corona discharges. For example, certain disclosed embodiments concern a microreactor comprising plural, simultaneously emitting emitter electrodes and a counter electrode. The electrodes may be arranged in series or parallel. These embodiments may be configured to produce plural, simultaneously operating corona discharges. Accordingly, certain disclosed microreactors comprise 1 to 100 simultaneously active corona discharges, more typically 3 to 50 simultaneously active corona discharges, such as 4 to 20 simultaneously active corona discharges.

Certain embodiments use a needle tip electrode, which typically is the negative electrode. A person of ordinary skill in the art will appreciate that the needle tip electrode need not be made from any particular metal or material. Suitable electrodes are typically made of a material that is sufficiently conductive to allow passage of tens of mA with minimal voltage drop. Furthermore, the selected electrode material preferably should have a large secondary electron emission yield (related to work function and other properties) and also resist electron driven sputtering of the electrode material. These two issues, discharge generation potential and robustness of the electrode material, are potentially inversely related, and hence each may be considered separately to optimize performance for a particular application. Accordingly, suitable electrodes may be made from a metal, metal alloy, or semiconductor. For certain exemplary embodiments the electrode material may be a metal or metal alloy comprising iron, nickel, palladium, platinum, tungsten, or combinations thereof. The electrodes also preferably are made of a material that can withstand occasional temperature excursions, so refractory metals and alloys comprising tungsten, and Ni superalloys, like Inconel, are suitable electrode materials, but common stainless steel and galvanized steel also may be used. Exemplary semiconductors suitable for forming electrodes include ZnO, LaB₆, and CeO. Materials like graphite, carbon felt, silicon and doped silicon also may work well. In order to enhance emission current and increase process efficiency, coated electrodes may be used. For example, electrodes may be coated with a low work function material, like ZnO, BaO or ThO₂ to enhance performance. Composite electrodes also can be used, such as metal/ceramic composites. A particular disclosed working embodiment comprised plural, simultaneously active corona discharge reactors arranged in series comprising plural emitter electrodes positioned and supported by an emitter support member that were operatively associated with a metal plate counter electrode.

The microreactor also includes a power supply that provides both the power and voltage requirements required to produce a corona discharge, as well as the ability to carefully control the input power to the reactor system. Suitable power supplies allow the ability to rapidly shape the power to stabilize the non-thermal DC plasma, rather than ending up in arc discharge (thermal) mode. Fast DC power supplies, such as a boost converter, interfaced with a suitable control (whether a digital PLC or an analogue circuit), are suitable for providing the requisite power and voltage requirements. Certain disclosed embodiments include a power supply having one or more inline ballast resistors, such as a 60 MΩ ballast resistor for carbon dioxide and an 80 MΩ ballast resistor for air.

A microreactor may be housed in a housing that defines at least one reactant feed inlet fluidly coupled to at least one corona discharge reactor. The microreactor typically has at least one feature measured on a millimeter scale, such as at least one fluid channel having at least one dimension of from about 1 to about 100 mm. Certain embodiments minimize fluid bypass in an active area of corona discharge by using a microchannel having a width, depth or length that is substantially occupied by an active area of corona discharge.

For certain embodiments, emitter electrodes were shaped like a cross. Each emitter needle resided in a purposefully formed cavity in a reactor plate that also defined a reaction microchannel for channeling a fluid to a corona discharge region.

Certain disclosed microreactors comprised multiple reactor plates in a stack. Each plate comprised plural corona discharge electrodes positioned in series along each of plural corresponding microchannels. The reactor plates may be made from any suitable material, such as a metal, an alloy, a phenolic resin, a castable ceramic, and combinations thereof. Certain disclosed embodiments used a glass-filled phenolic resin, a glass-filled silicone resin, or combinations thereof. A particular embodiment used reactor plates made from Portland Cement. Reactor features, such as microchannels and electrode receiving portion, were formed in the Portland Cement plate by stamping.

A reactant distribution manifold may be operatively associated with the reactor stack to distribute reactants to reactant inlet ports. A product distribution manifold may be operatively associated with product outlet ports to receive product produced by the reactor and to distribute such product to downstream components for further processing or product collection in a reservoir. As will be understood by a person of ordinary skill in the art, disclosed microreactors are useful for processing fluids, most typically gases. Accordingly, disclosed reactors often include at least one fluid seal to provide a fluidly sealed device and to potentially define microchannels. And, the microreactor also typically includes a power supply coupled to the electrodes.

Discharge microreactor systems also are disclosed. Certain embodiments comprise at least one microreactor configurable to produce a corona discharge, the microreactor comprises plural, emitter electrodes capable of operating in series and at least one counter electrode. The electrodes are configured to produce plural, simultaneously operating corona discharges. The system includes a power supply coupled to the electrodes. Certain power supplies were configured for transition from high voltage/low current (˜2 kV/1 nA) to lower voltage/higher current (˜300V/100 mA) Disclosed microreactor systems may optionally further comprise at least one of: a computer to control operating parameters, data acquisition, or both; product analytic instrumentation, such as a gas chromatograph, a mass spectrometer, an FT-IR spectrometer, a Raman gas analyzer, and combinations thereof; a pressure regulator; a condenser; a fluid inlet distribution manifold; a product distribution manifold; a fluid product collection reservoir; a heat exchanger; a pressure transmitter; a temperature transmitter; or combinations thereof.

A particular embodiment of a microreactor system according to the present invention includes plural gas reactant sources selected from methane (CH₄), nitrogen (N₂), carbon dioxide (CO₂) and water (H₂O). Mass flow controllers were associated with each of the plural gas reactant sources to flow a predetermined amount of a fluid reactant either to a corona discharge microreactor or system comprising a disclosed microreactor, or to a mixer for receiving plural gas reactants to form mixtures that are flowed through a corona discharge reactor.

One particular embodiment of a microreactor was designed to produce C₂ or greater hydrocarbons, such as C₂-C₁₆ hydrocarbons, by condensing methane. These embodiments included a corona discharge reactor configured for converting biogas to higher condensed hydrocarbons; a source of biogas; a pressure regulator to control reactor inlet pressure; a DC power source coupled to the reactor and controlled by a power supply control unit; a heat exchanger to facilitate recovering condensable products generated by the reactor as liquids; a liquids collection reservoir; and a back pressure regulator to control reactor exit pressure.

Embodiments of a method for using disclosed microreactors and systems comprising disclosed microreactors also are disclosed. For example, the method may be used for chemical transformations, fluid purifications, or both. One particular embodiment concerns producing C₂ or greater hydrocarbons, preferably for producing gasoline comprising a hydrocarbon chain of about C₁₀ or diesel comprising a hydrocarbon chain of about C₁₆, from a reactant stream comprising methane. A particular embodiment comprises providing a reactant flow of biogas from a dairy to a microreactor comprising plural, simultaneously operating corona discharge regions, or to a reactor stack comprising plural corona discharge microreactors, to produce C₂ or greater hydrocarbons. Other disclosed method embodiments include, without limitation: fluid purification, such as desulfurization of a hydrocarbon stream; and subjecting carbon dioxide in a reactant stream to reduce carbon dioxide concentration.

The foregoing and other objects, features, and advantages of the invention will become more apparent from the following detailed description, which proceeds with reference to the accompanying figures.

### BRIEF DESCRIPTION OF THE DRAWINGS

FIG. 1 is a schematic drawing of one disclosed embodiment of a flow-through plasma microreactor system, where FIG. 1A is prior to operation with an unused Ni tip electrode, and FIG. 1B illustrates steady glow discharge in 3:1 CH₄:O₂ flow at 40 mL/minute with a sustaining voltage of 600 volts and 2.1 mA discharge current after several hours operation time.

FIG. 2 is an image photograph of a reactor operating in a steady state glow discharge just after ignition.

FIG. 3 is an image of a reactor operating at higher currents relative to the reactor of FIG. 2 and prior to transition to an arc discharge.

FIG. 4 is a circuit diagram of one embodiment of a reactor according to the present invention comprising 3 simultaneously active corona discharges.

FIG. 5 is an image of an operating reactor having 3 simultaneously active corona discharges.

FIG. 6 is a schematic diagram illustrating one embodiment of a multiple tandem discharge microreactor according to the present invention.

FIG. 7 is a longitudinal cross sectional view of one embodiment of a multiple tandem discharge microreactor according to the present invention.

FIG. 8 is a transverse cross sectional view of one embodiment of a multiple tandem discharge microreactor according to the present invention.

FIG. 9 is a schematic drawing of one embodiment of an emitter needle according to the present invention.

FIG. 10 is a schematic diagram of one embodiment of a reactor tile of a corona discharge microreactor according to the present invention comprising plural cross-shaped housings to house plural self-centering emitter needles, and plural longitudinal reaction microchannels associated with serially positioned emitter electrodes.

FIG. 11 is a graph of current (mA) versus reactor electrical potential (V) for various different electrode placement distances.

FIG. 12 is a schematic perspective view of one embodiment of a reactor stack according to the present invention comprising 10+1 reactor plates, a sealing plate, reactant inlets on the proximal face, product outlets on the posterior face (not shown), a first set of electrode connections on the right side of the stack and a second set of electrodes (not shown) on the left side of the stack.

FIG. 13 is a schematic perspective drawing of a reactor and housing according to one embodiment of the present invention.

FIG. 14 illustrates a case and push connector for coupling external fluid flow lines to reactor housings according to the present invention.

FIG. 15 is an image of a multi-discharge reactor according to the present invention.

FIG. 16 is an image of a multi-discharge reactor according to the present invention.

FIG. 17 is a schematic diagram of one system comprising a reactor for determining products produced by subjecting various reaction streams comprising CO₂ to corona discharge.

FIG. 18 is a schematic drawing of a corona-discharge reactor system according to the present invention.

FIG. 19 is a schematic diagram of a system comprising a corona discharge reactor according to the present invention, wherein the system is useful for converting biogas to higher condensed hydrocarbons.

FIG. 20 is a graph of current (mA) versus power (W) showing the control applied power as a function of current.

FIG. 21 is a graph of current (mA) versus voltage (V) showing the resulting voltage profile needed to allow current flow, where the vertical dashed line shows the conceptual transition from a pulsed discharge to a steady state glow/corona discharge.

FIG. 22 is a graph of power (W) versus conversion or selectivity (%), illustrating conversion of a stream of methane passing through a corona discharge reactor as a function of applied power, and the selectivity of the converted stream to produce ethylene (squares) and acetylene (triangles), with larger molecules forming the balance. FIG. 22 illustrates initial low conversion at low power, since this takes place in a spark discharge regime characterized by very short duration emission of a few extremely energetic electrons. As power increases stable glow discharge is established, characterized by higher currents (i.e., higher electron flux) at lower voltages (i.e., less energy per electron, resulting in more thorough use of the energy of each electron).

FIG. 23 is a graph of methane feed percentage versus product percentage illustrating product distribution of products produced by subjecting methane/oxygen feed streams to corona discharge reactors, where the reactant feed composition ranged from a 50/50 mixture to pure methane feed gas (while electrical properties vary slightly with feed gas composition, typical operating conditions are approximately 500 V sustaining voltage and 1.5 mA discharge current for discharge power around 1 W).

FIG. 24 is a schematic drawing illustrating an exemplary embodiment of a chemical transformation method according to the present invention.

FIG. 25 provides gas chromatography product analysis results for products produced by subjecting various reaction streams comprising CO₂ to corona discharge using a corona discharge reactor having three discharge regions in series.

FIG. 26 is a graph qualitatively illustrating molar inlet and outlet fluxes produced by a corona discharge reactor having three discharge regions in series wherein the reactor receives reactant inlet feed streams comprising CO₂.

FIG. 27 is a graph of year versus atmospheric CO₂ concentration in parts per million from 1958 to 2015.

FIG. 28 is a bar graph illustrating conversion of CO₂ using a corona discharge reactor having three discharge regions in series according to the present invention.

FIG. 29 is a bar graph illustrating the effect of power on efficiency for CO₂ reduction using a corona discharge reactor according to the present invention.

FIG. 30 is a bar graph illustrating energy efficiency for CO₂ reduction using a corona discharge reactor according to the present invention at various different feed (20, 50 and 50 standard cubic centimeters per minute rates (sccm)).

FIG. 31 is a bar graph illustrating percent conversion to products for CO₂ reduction using a corona discharge reactor according to the present invention at various different feed (20, 50 and 50 standard cubic centimeters per minute rates (sccm)).

FIG. 32 provides an energy analysis for the production of methane, carbon monoxide and oxygen from carbon dioxide and water.

FIG. 33 provides an exergy analysis for exergy efficiency.

FIG. 34 is an exploded perspective view of an embodiment of a corona-discharge microreactor according to the present invention.

FIG. 35 is a perspective view of a reactor plate with a first channel and a second channel according to the present invention.

### DETAILED DESCRIPTION

### I. Definitions

The following explanations of terms and methods are provided to better describe the present disclosure and to guide those of ordinary skill in the art in the practice of the present disclosure. The singular forms “a,” “an,” and “the” refer to one or more than one, unless the context clearly dictates otherwise. The term “or” refers to a single element of stated alternative elements or a combination of two or more elements, unless the context clearly indicates otherwise. As used herein, “comprises” means “includes.” Thus, “comprising A or B,” means “including A, B, or A and B,” without excluding additional elements. All references, including patents and patent applications cited herein, are incorporated by reference.

Unless otherwise indicated, all numbers expressing quantities of components, molecular weights, percentages, temperatures, times, and so forth, as used in the specification or claims are to be understood as being modified by the term “about.” Accordingly, unless otherwise indicated, implicitly or explicitly, the numerical parameters set forth are approximations that may depend on the desired properties sought and/or limits of detection under standard test conditions/methods. When directly and explicitly distinguishing embodiments from discussed prior art, the embodiment numbers are not approximates unless the word “about” is expressly recited.

Unless explained otherwise, all technical and scientific terms used herein have the same meaning as commonly understood to one of ordinary skill in the art to which this disclosure pertains. Although methods and materials similar or equivalent to those described herein can be used in the practice or testing of the present disclosure, suitable methods and materials are described below. The materials, methods, and examples are illustrative only and not intended to be limiting.

When chemical structures are depicted or described, unless explicitly stated otherwise, all carbons are assumed to include hydrogen so that each carbon conforms to a valence of four. For example, in the structure on the left-hand side of the schematic below there are nine hydrogen atoms implied. The nine hydrogen atoms are depicted in the right-hand structure.

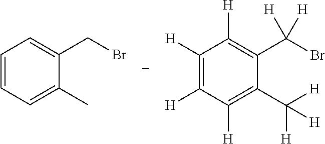

Sometimes a particular atom in a structure is described in textual formula as having a hydrogen or hydrogen atoms, for example —CH₂CH₂—. It will be understood by a person of ordinary skill in the art that the aforementioned descriptive techniques are common in the chemical arts to provide brevity and simplicity to description of organic structures.

If a group R is depicted as “floating” on a ring system, as for example in the group:

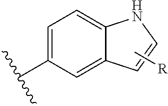

then, unless otherwise defined, a substituent R can reside on any atom of the fused bicyclic ring system, excluding the atom carrying the bond with the “ [symbol] ” symbol, so long as a stable structure is formed. In the example depicted, the R group can reside on an atom in either the 5-membered or the 6-membered ring of the indolyl ring system.

Aliphatic: A substantially hydrocarbon-based compound, or a radical thereof (e.g., C₆H₁₃, for a hexane radical), including alkanes, alkenes, alkynes, including cyclic versions thereof, and further including straight- and branched-chain arrangements, and all stereo and position isomers as well. Unless expressly stated otherwise, an aliphatic group contains from one to twenty-five carbon atoms; for example, from one to fifteen, from one to ten, from one to six, or from one to four carbon atoms. The term “lower aliphatic” refers to an aliphatic group containing from one to ten carbon atoms. An aliphatic chain may be substituted or unsubstituted. Unless expressly referred to as an “unsubstituted aliphatic,” an aliphatic group can either be unsubstituted or substituted. An aliphatic group can be substituted with one or more substituents (up to two substituents for each methylene carbon in an aliphatic chain, or up to one substituent for each carbon of a —C═C— double bond in an aliphatic chain, or up to one substituent for a carbon of a terminal methine group). Exemplary substituents include, but are not limited to, alkyl, alkenyl, alkynyl, alkoxy, alkylamino, alkylthio, acyl, aldehyde, amide, amino, aminoalkyl, aryl, arylalkyl, carboxyl, cyano, cycloalkyl, dialkylamino, halo, haloaliphatic, heteroaliphatic, heteroaryl, heterocycloaliphatic, hydroxyl, oxo, sulfonamide, sulfhydryl, thioalkoxy, or other functionality.

Alkyl: A hydrocarbon group having a saturated carbon chain. The chain may be cyclic, branched or unbranched. Examples, without limitation, of alkyl groups include methyl, ethyl, propyl, butyl, pentyl, hexyl, heptyl, octyl, nonyl and decyl. The term lower alkyl means the chain includes 1-10 carbon atoms. The terms alkenyl and alkynyl refer to hydrocarbon groups having carbon chains containing one or more double or triple bonds, respectively.

Anode: An electrode through which electric charge flows into a polarized electrical device. From an electrochemical point of view, negatively-charged anions move toward the anode and/or positively-charged cations move away from it to balance the electrons leaving via external circuitry. In a discharging battery or galvanic cell, the anode is the negative terminal where electrons flow out. If the anode is composed of a metal, electrons that it gives up to the external circuit are accompanied by metal cations moving away from the electrode and into the electrolyte. When the battery is recharged, the anode becomes the positive terminal where electrons flow in and metal cations are reduced.

Bis: A prefix meaning “twice” or “again.” It is used in chemical nomenclature to indicate that a chemical group or radical occurs twice in a molecule. For example, a bis-ester has two ester groups.

Cathode: An electrode through which electric charge flows out of a polarized electrical device. From an electrochemical point of view, positively charged cations invariably move toward the cathode and/or negatively charged anions move away from it to balance the electrons arriving from external circuitry. In a discharging battery or galvanic cell, the cathode is the positive terminal, toward the direction of conventional current. This outward charge is carried internally by positive ions moving from the electrolyte to the positive cathode, where they may be reduced. When the battery is recharged, the cathode becomes the negative terminal where electrons flow out and metal atoms (or cations) are oxidized. Presently disclosed embodiments are not electrochemical devices, and hence the cathode is the electrode through which energetic electrons are injected into a non-thermal plasma (electrons flowing in =“positive charge flows out” according to electrical circuit convention).

Contacting: Placement that allows association between two or more moieties, particularly direct physical association, for example the placement of a reactant stream, particularly in a gas phase, to contact a corona discharge.

Free radical: An atom, molecule, or ion with an unpaired electron. Free radicals are formed by splitting a chemical bond within a molecule, and are usually short-lived and highly reactive. Free radicals are capable of initiating chemical chain reactions, e.g., dimerization and polymerization. They also act as initiators or intermediates in oxidation, combustion, and photolysis.

Functional group: A specific group of atoms within a molecule that is responsible for the characteristic chemical reactions of the molecule. Exemplary functional groups include, without limitation, alkyl, alkenyl, alkynyl, aryl, halo (fluoro, chloro, bromo, iodo), epoxide, hydroxyl, carbonyl (ketone), aldehyde, carbonate ester, carboxylate, carboxyl, ether, ester, peroxy, hydroperoxy, carboxamide, amino (primary, secondary, tertiary), ammonium, imide, azide, cyanate, isocyanate, thiocyanate, nitrate, nitrite, nitrile, nitroalkyl, nitroso, pyridyl, phosphate, sulfonyl, sulfide, thiol (sulfhydryl), disulfide.

Hydrocarbon: An organic compound consisting of the elements carbon and hydrogen. Hydrocarbons include aliphatic compounds (alkanes, alkenes, alkynes, and cyclic versions thereof, including straight- and branched-chain arrangements), aromatic compounds (unsaturated, cyclic hydrocarbons having alternate single and double bonds), and combinations thereof (e.g., arylalkyl compounds). Hydrocarbons can be produced according to certain disclosed embodiments of the present invention.

Isomer: One of two or more molecules having the same number and kind of atoms, but differing in the arrangement or configuration of the atoms. Isomers that differ in the arrangement of their atoms in space are termed “stereoisomers”. Stereoisomers that are not mirror images of one another are termed “diastereomers” and those that are non-superimposable mirror images of each other are termed “enantiomers.” When a compound has an asymmetric center, for example, if a carbon atom is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−) isomers respectively). A chiral compound can exist as either individual enantiomer or as a mixture thereof. A mixture containing equal proportions of the enantiomers is called a “racemic mixture.” E/Z isomers are isomers that differ in the stereochemistry of a double bond. An E isomer (from entgegen, the German word for “opposite”) has a trans-configuration at the double bond, in which the two groups of highest priority are on opposite sides of the double bond. A Z isomer (from zusammen, the German word for “together”) has a cis-configuration at the double bond, in which the two groups of highest priority are on the same side of the double bond. The E and Z isomers of 2-butene are shown below:

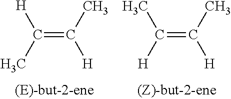

For embodiments of the present invention where products can be made having sites of unsaturation, such products are understood to include all possible isomers, including all stereoisomers and E/Z isomers, unless expressly stated otherwise or the context would be understood by a person of ordinary skill in the art to include or exclude a certain isomer or isomers.

Lower: Refers to organic compounds having 10 or fewer carbon atoms in a chain, including all branched and stereochemical variations, particularly including methyl, ethyl, propyl, butyl, pentyl, hexyl, heptyl, octyl, nonyl, and decyl.

Moiety: A moiety is a fragment of a molecule, or a portion of a conjugate.

Molecular weight: The sum of the atomic weights of the atoms in a molecule. As used herein with respect to polymers, the terms molecular weight, average molecular weight, and mean molecular weight refer to the number-average molecular weight, which corresponds to the arithmetic mean of the molecular weights of individual macromolecules. The number-average molecular weight may be determined by any method generally known by persons of ordinary skill in the art, such as chromatographic methods.

Monomer: A molecule or compound, usually containing carbon, that can react and combine to form polymers. Molecules formed by the combination of monomers can be characterized by the number of monomers. For example, a dimer is a molecule formed from two monomers, a trimer is a molecule formed from three monomers, etc. A molecule with more than 10 monomers is typically referred to using the number of monomeric units, e.g., a 20-mer is a molecule having 20 monomeric units. As may be established by mass spectrometry, proton NMR, carbon NMR, methods such as vapor pressure osmometry and end group assays, etc., and combinations thereof.

Olefin: An unsaturated aliphatic hydrocarbon having one or more double bonds. Olefins with one double bond are alkenes; olefins with two double bonds are alkadienes or diolefins.

Precursor: An intermediate compound or molecular complex. A precursor participates in a chemical reaction to form another compound.

Providing a compound or composition comprising the compound: Refers to a person, entity or other manufacturer who makes the compound or composition comprising the compound and provides instructions for its use, such as by establishing the manner and/or timing of using the compound or composition; a supplier who supplies the compound or composition and provides instructions for its use, establishing the manner and/or timing of using the compound or composition; a facility that uses the compound or composition; and/or a subject who uses the compound or composition themselves. The manufacturer, supplier, facility and/or subject may act jointly or as a joint enterprise by agreement, by a common purpose, a community of pecuniary interest, and/or substantially equal say in direction of using the compound or composition. Alternatively, or additionally, the manufacturer, supplier, facility and/or subject may condition participation in an activity or receipt of a benefit upon performance of a step or steps of the method of using the compound or composition disclosed herein, and establish the manner and/or timing of that performance.

Stereoisomers: Isomers that have the same molecular formula and sequence of bonded atoms, but which differ only in the three-dimensional orientation of the atoms in space.

Structural unit: As used herein, the term “structural unit” refers to a unit of a polymer derived from polymerization of monomers.

Substituent: An atom or group of atoms that replaces another atom in a molecule as the result of a reaction. The term “substituent” typically refers to an atom or group of atoms that replaces a hydrogen atom, or two hydrogen atoms if the substituent is attached via a double bond, on a parent hydrocarbon chain or ring. The term “substituent” may also cover groups of atoms having multiple points of attachment to the molecule, e.g., the substituent replaces two or more hydrogen atoms on a parent hydrocarbon chain or ring. In such instances, the substituent, unless otherwise specified, may be attached in any spatial orientation to the parent hydrocarbon chain or ring. Exemplary substituents include, for instance, alkyl, alkenyl, alkynyl, alkoxy, alkylamino, alkylthio, acyl, aldehyde, amido, amino, aminoalkyl, aryl, arylalkyl, arylamino, carbonate, carboxyl, cyano, cycloalkyl, dialkylamino, halo, haloaliphatic (e.g., haloalkyl), haloalkoxy, heteroaliphatic, heteroaryl, heterocycloaliphatic, hydroxyl, oxo, sulfonamide, sulfhydryl, thio, and thioalkoxy groups.

Substituted: A fundamental compound, such as an aryl or aliphatic compound, or a radical thereof, having coupled thereto one or more substituents, each substituent typically replacing a hydrogen atom on the fundamental compound. Solely by way of example and without limitation, a substituted aryl compound may have an aliphatic group coupled to the closed ring of the aryl base, such as with toluene. Again solely by way of example and without limitation, a long-chain hydrocarbon may have a hydroxyl group bonded thereto. “Substituted” refers to all subsequent modifiers in a term, for example in the term “substituted arylC₁₋₈ alkyl,” substitution may occur on the “C₁₋₈alkyl” portion, the “aryl” portion or both portions of the arylC₁₋₈ alkyl group. “Substituted,” when used to modify a specified group or moiety, means that at least one, and perhaps two or more, hydrogen atoms of the specified group or moiety is independently replaced with the same or different substituent groups. In a particular embodiment, a group, moiety or substituent may be substituted or unsubstituted, unless expressly defined as either “unsubstituted” or “substituted.” Accordingly, any of the groups specified herein may be unsubstituted or substituted. In particular embodiments, the substituent may or may not be expressly defined as substituted, but is still contemplated to be optionally substituted. For example, an “alkyl” or a “pyrazolyl” moiety may be unsubstituted or substituted, but an “unsubstituted alkyl” or an “unsubstituted pyrazolyl” is not substituted.

Sulfonyl: A functional group with the general formula:

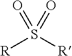

where R and R′ independently are selected from various groups, including by way of example aliphatic, substituted aliphatic, cyclic aliphatic, substituted cyclic aliphatic, aryl, substituted aryl, heteroaryl, and substituted heteroaryl.

### II. General Discussion

Plasmas generated by electrical discharges are known and have been used to attempt chemical transformations, including reforming hydrocarbon on board vehicles. For methane conversion the major process previously applied appears to have been Dielectric Barrier Discharge instead of direct plasma discharge, most probably due to difficulties associated with controlling a stable corona plasma discharge at macroscopic dimensions. Dielectric Barrier Discharge accelerates heavy particles in an AC field causing multiple ion-ion collisions. Both Dielectric Barrier Discharge and Arc Discharge result in much higher temperatures and produce thermal plasmas, whereas a corona discharge avoids these collisions and results in low temperatures and cold plasmas. For at least this reason, corona discharges are intrinsically more energy efficient than Dielectric Barrier Discharge and Arc Discharge.

One aspect of the present invention is to control the corona discharge using a microreactor comprising one or more purposefully configured emitter electrodes, such as needle-like emitter electrodes having a short anode-cathode gap, and/or a power supply configured to precisely control voltage and power to the electrodes. A microreactor is a relatively small device having at least one operative feature having at least one dimension that is a few millimeters or tenths of millimeters in size, generally from about 1 mm to about 1000 millimeters, and more typically from about 5 to 500 millimeters, in size. More typically, disclosed microreactors or microchannel reactors have at least one sub-1 mm dimension. For example, a microreactor may have one or plural fluid microchannels, wherein at least one microchannel has at least one dimension a few millimeters or tenths of millimeters in size, generally from about 1 mm to about 1000 millimeters, and more typically from about 5 to 500 millimeters. As another example, a gap defined between an emitter electrode and counter electrode may be measured in millimeters or tenths of millimeters. Certain disclosed microreactor embodiments had a gap size of from about 0.1 mm to about 10 mm, such as from about 0.1 mm to about 5 mm, even more typically from about 0.1 to 2 mm, or greater than 0 to less than 1 mm. A person of ordinary skill in the art will appreciate that it becomes more difficult to establish a corona discharge as the gap size increases towards a 10 mm gap. Establishing a stable corona discharge at a 2 mm scale is relatively facile, but becomes more difficult at a 10 mm gap, as the discharge may result in an arc discharge.

Disclosed microreactors that can be assembled as arrays of microreactors operating either in series, in parallel, or both. Plural microreactors also may be provided as an array on a reactor plate. Plural such reactor plates may be assembled to produce a reactor stack.

Disclosed electrical discharge microreactors can be operated in a number of different modes, including in a pulse discharge mode, an arc discharge mode, and/or a corona discharge mode. For example, a particular disclosed corona discharge microreactor had a gap size of about 500 μm, had an applied voltage of 500 volts and operated at about 5 mA. This reactor operated as an arc discharge device with the same applied 500 volts at 10 to 20 mA. In general, the greater the gap distance between the electrodes, the greater the applied voltage required to induce a corona discharge. Arc discharge produces substantial thermal energy, but most of the thermal energy generated is not usefully applied to, for example, induce chemical transformations. In contrast, a corona discharge does not generate substantial thermal energy. Most useful energy produced by a corona discharge is provided by the kinetic energy of electrons. Non-thermal corona discharges are generally characterized by low gas temperatures, but high electron temperatures, with high concentrations of free electrons (up to a density of 10¹⁹ m⁻³). Certain disclosed corona discharge reactors comprise an atmospheric, non-thermal plasma that is created by electrical discharge initiated by ionization of a fluid surrounding an electrically energized conductor.

A corona discharge may be produced by applying a relatively high voltage to a point emitter, typically a negative electrode, which is associated with a counter electrode, typically a positive electrode, which can be provided as a point or surface, such as a plate electrode. A non- or poorly-conductive gas is present in the gap defined between the emitter electrode and the counter electrode. The gap gas generally is non- or poorly conductive, and it takes a relatively high voltage to induce electron streaming. If the voltage is not high enough, then the gas in the gap tends to ionize. Suitable voltages for disclosed embodiments are from about 1600 volts to about 2300 volts. If voltage is increased above this value, then electrical breakdown of the gap gas occurs. As soon as the gap gas undergoes electrical breakdown, electrons stream from a negative emitter electrode to a positive counter electrode, and the gap is now filled with a conductive gas. Substantial current can now flow because the gas-filled space is rendered conductive. The color of the corona discharge depends on the composition of the gap gas. For example, air and nitrogen produce a purple corona discharge; carbon dioxide produces a bluish-white corona discharge.

Disclosed microreactors are useful for a number of processes including, by way of example and without limitation: chemical transformations, such as converting carbon dioxide in an inlet feed stream to reduce carbon dioxide emissions or converting methane and other short hydrocarbons to longer-chain hydrocarbons including condensed liquids for use as liquid fuels; decontamination of fluids, such as desulfurization of hydrocarbons and production of potable or highly purified water; and combinations of these processes. One exemplary embodiment concerns the condensation of natural gas to liquids using a non-thermal electric corona plasma discharge. Using a non-thermal corona discharge process that does not require high temperatures for the desired conversion will naturally lead to lower energy losses, since no process heat is required to achieve the conversion.

Corona discharge typically is induced in a gas phase, and certain exemplary disclosed embodiments concern conducting chemical transformations in a gas phase. However, corona discharge reactions also can be applied to a liquid phase. Typically, the corona discharge is first induced in a gas phase, and then the discharge is applied to the liquid phase and/or to components dissolved or suspended in the liquid phase.

Corona discharge provides substantial benefits relative to known processes. For example, corona discharge approaches 90% energy efficiency for conducting chemical transformations, such as the production of longer chain, liquid hydrocarbons from lighter gas reactants, such as methane (CH₄). This compares to 50% or less energy efficiency for electrolytic processes used to make the same compounds.

### III. Corona Discharge Reactor and Systems Comprising One or More Reactors

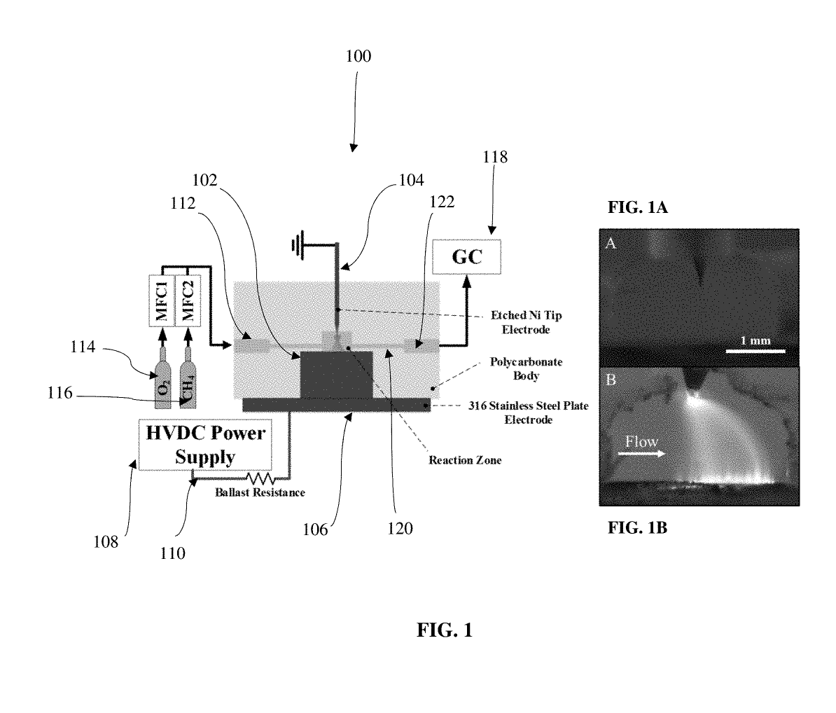

FIG. 1 is a schematic drawing of one disclosed embodiment of a flow-through plasma microreactor system 100. System 100 includes one embodiment of a corona discharge reactor 102 comprising a single emitter electrode 104 and a plate counter electrode 106. FIG. 1A illustrates the reactor prior to operation with an unused Ni tip electrode. FIG. 1B illustrates steady glow discharge in 3:1 CH₄:O₂ flow at 40 mL/minute with a sustaining voltage of 600 volts and 2.1 mA discharge current after several hours operation time. System 100 further includes a power supply 108 having an inline ballast resistance 110. Reactant fluid inlet 112 is fluidly associated with inlet feed reactants, such as the illustrated oxygen (O₂) and methane (CH₄) fluid canisters 114 and 116. System 100 also may include a gas chromatograph (GC) 118 for receiving at least a portion of a fluid product stream 120 from outlet 122.

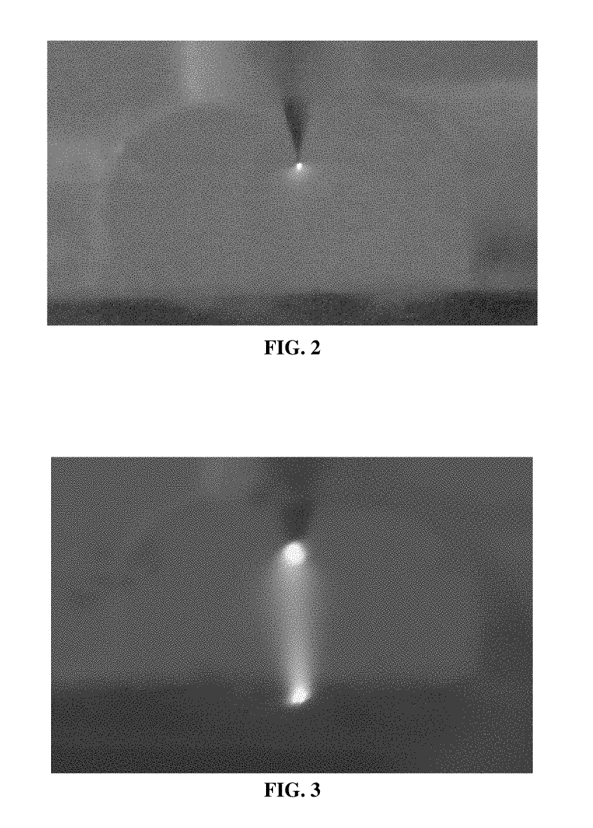

FIG. 2 is an image illustrating the appearance of a single discharge just past ignition (i.e., at the lowest power level that maintains the plasma stable). FIG. 3 is an image of a discharge in pure oxygen illustrating the appearance of a single corona discharge at the highest threshold (before transition to an arc discharge, which transition is noticeable by a sharp increase in voltage/power to achieve a marginal increase in current). In the reactor from which these images were captured gas flows in the direction transverse to the electric current (i.e., left to right in the figures).

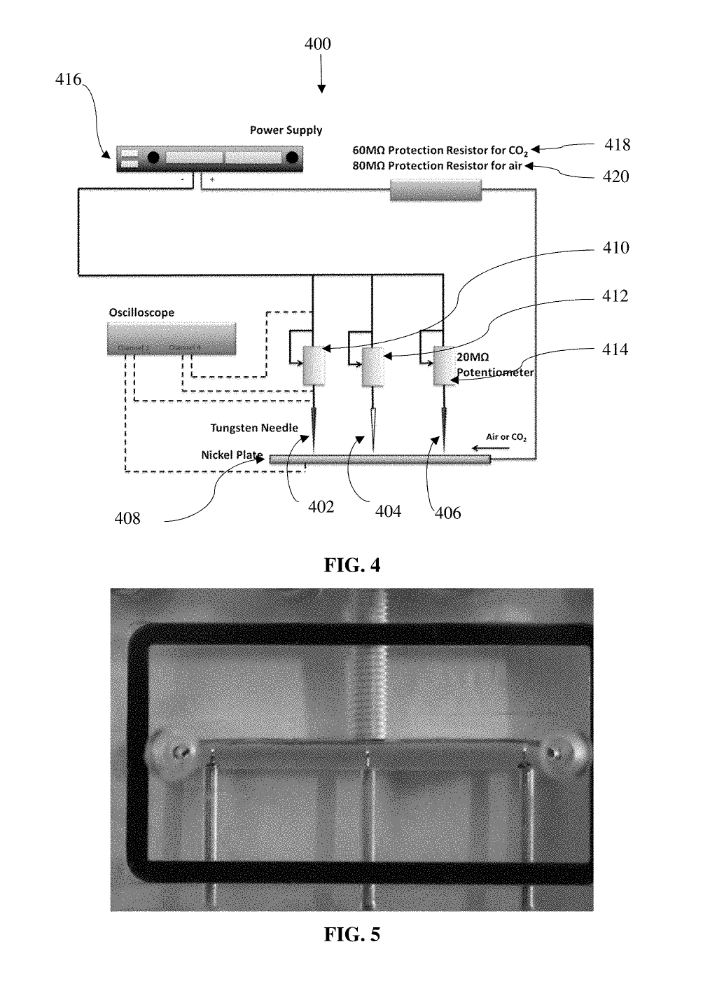

Plural discharges can be produced in a single reactor. FIG. 4 is a circuit diagram of one embodiment of a reactor system 400 according to the present invention comprising 3 simultaneously active corona discharges. System 400 includes three tungsten needle emitter negative electrodes 402, 404 and 406 arranged in series, although a person of ordinary skill in the art will understand that the electrodes could be arranged in series, in parallel, or a device may include a region with plural electrodes arranged in series and at least one other region where plural electrodes are arranged in parallel. Emitter electrodes 402, 404 and 406 are associated with a counter electrode, such as a nickel-plated counter (typically positive) electrode 408. The illustrated embodiment 400 includes optional monitoring components. For example, the illustrated embodiment includes, coupled to each emitter electrode, a 20 MΩ potentiometer 410, 412, and 414. Emitter electrodes 402, 404 and 406 are coupled to power supply 416, which may include an inline ballast resistor. A particular disclosed embodiment included a 60 MΩ ballast resistor 418 for carbon dioxide and an 80 MΩ ballast resistor 420 for air.

FIG. 5 is an image of an operating reactor comprising 3 simultaneously active corona discharges ignited by a single power supply in a single reactor. Disclosed embodiments may have more than 3 simultaneously active corona discharges in a single reactor, such as a single reactor having from 4 to 100, more typically 4 to 50, even more typically 4-20, such as 4-10, simultaneously active corona discharges.

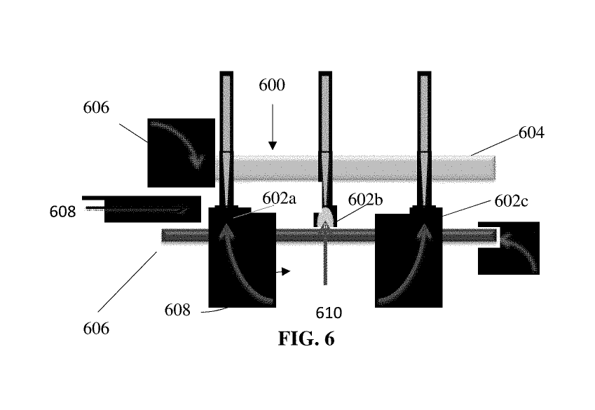

FIG. 6 illustrates one embodiment of a reactor 600 comprising plural, simultaneously active corona discharge reactors arranged in series. Reactor 600 includes plural emitter electrodes 602a, 602b and 602c positioned and supported in the device 600 using emitter support member 604. Reactor 600 also includes a metal plate 606 as a counter electrode to the emitter electrodes 602. Reactor 600 also defined a channel 608 for receiving a flow of reactant gas and flowing the reactant(s) to an active area of corona discharge 610. For certain embodiments, the channel 608 ranged from a width of from about 50 to about 500 μm and the discharge region 610 had a diameter of about 1 mm. Accordingly, the dimensions of one disclosed embodiment of a reactor 600 includes a reactor gas fluidic channel 608 having at least one dimension, such as width and/or depth, of about 1 mm to substantially encompass the active area of corona discharge, thereby minimizing gas bypass around the discharge region. If a channel is too broad, then reactant-to-product conversions percentages may be reduced as the channel dimensions increase. And, if reactor dimensions are too small, electrons may repeatedly strike previously activated gas molecules, increasing energy waste. Specifically, reactor dimensions are preferably appropriately scaled to the size of the discharge (i.e. active plasmachemical area). This dimension may change depending on reactor design. For certain working embodiments, the reactive area was approximately cylindrical with a diameter of about 400 microns. Accordingly, for these embodiments, reactor dimensions of from about 0.05 mm to about 1 mm are acceptable. Moreover, disclosed reactor embodiments may include inclusions to guide gas flow solely to those reactor areas.

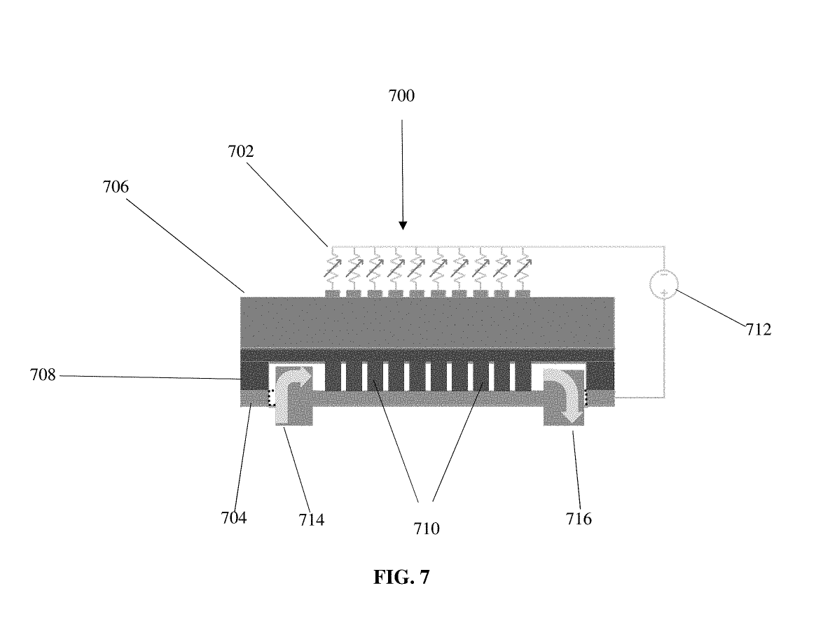

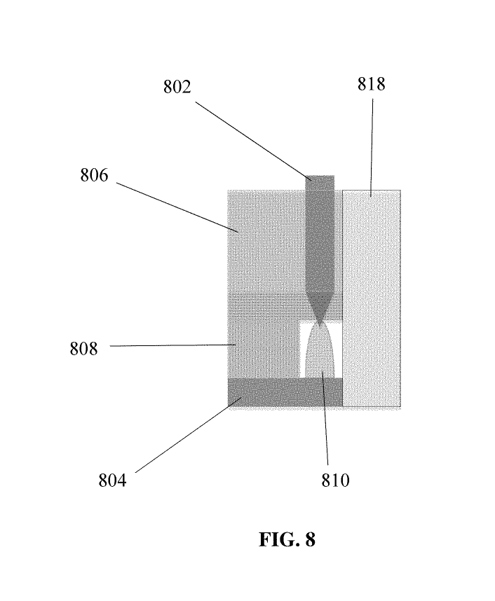

FIGS. 7 and 8 are longitudinal and transverse cross sections, respectively, of one embodiment of multiple tandem discharge microreactors. Microreactor 700 comprises plural emitter electrodes 702 and associated counter electrode 704 positioned to establish a corona discharge. For one disclosed embodiment, the emitter electrodes 702 are sharpened tungsten rods, but this choice of material was primarily to ensure that even if high temperatures occur at the emitter tips (e.g., if a discharge operated as an arc discharge) that no thermal damage takes place. The counter-electrode 704 may be fashioned as a plate, such as a metal or alloy plate, with a stainless steel plate being used for particular working embodiments. Emitter electrodes 702 are positioned appropriately by an emitter electrode support plate 706. A spacer 708, such as a silicone rubber spacer, will be used to create the fluidic channel within which the reaction may take place. Microreactor 700 produces plural, simultaneously operating corona discharges 710. Microreactor 700 also includes a DC power source 712 coupled to the electrodes. A fluid inlet, such as gas inlet 714, is provided to receive a flow of a reactant, or mixture of reactants, that is channeled to the corona discharges 710. Product produced by microreactor 700 flow from outlet 716 for collection or further processing.

FIG. 8 illustrates that a reactor may include at least one transparent wall, such as glass wall 818. Wall 818 allows a user to observe the discharge region during operation.

Certain disclosed embodiments of corona discharge microreactors include emitter needles as the negative electrode. A person of ordinary skill in the art will appreciate that the needle tip electrode need not be made from any particular metal or material. Suitable electrodes are typically made of a material that is sufficiently conductive to allow passage of tens of mA with minimal voltage drop. Furthermore, the selected electrode material preferably should have a large secondary electron emission yield (related to work function and other properties) and also resist electron driven sputtering of the electrode material. These two issues, discharge generation potential and robustness of the electrode material, are potentially inversely related, and hence each may be considered separately to optimize performance for a particular application. Accordingly, suitable electrodes may be made from a metal, metal alloy, or semiconductor. For certain exemplary embodiments the electrode material may be a metal or metal alloy comprising iron, nickel, palladium, platinum, tungsten, or combinations thereof. The electrodes also preferably are made of a material that can withstand occasional temperature excursions, so refractory metals and alloys comprising tungsten, and Ni superalloys, like Inconel, are suitable electrode materials, but common stainless steel and galvanized steel also may be used. Exemplary semiconductors suitable for forming electrodes include ZnO, LaB₆, and CeO. Materials like graphite, carbon felt, silicon and doped silicon also may work well. In order to enhance emission current and increase process efficiency, coated electrodes may be used. For example, electrodes may be coated with a low workfunction material, like ZnO, BaO or ThO₂ to enhance performance. Composite electrodes also can be used, such as metal/ceramic composites.

The microchannel and electrodes are designed to minimize reactant bypass. For certain working embodiments, the active area was approximately a cylinder with a diameter of 0.5 mm. For these embodiments, the electrodes may vary in size from about 0.05 mm to about 2 mm, particularly if the channel is designed so that the gas is forced to flow mainly through the active volume.

Emitter needles are positioned by a reactor tile relative to a ground plate to provide a discharge gap between the emitter and ground electrodes. Suitable gap distances are limited by practicality on the lower dimension, with a distance that is too short causing an active volume that is too small for practical gas conversion. The upper gap size is determined by a size that allows stabilizing a non-thermal plasma discharge at ambient pressure, with a probable upper limit of about a 10 mm scale, more likely a 5 mm scale, and even more typically having an upper limit of about 2 mm.

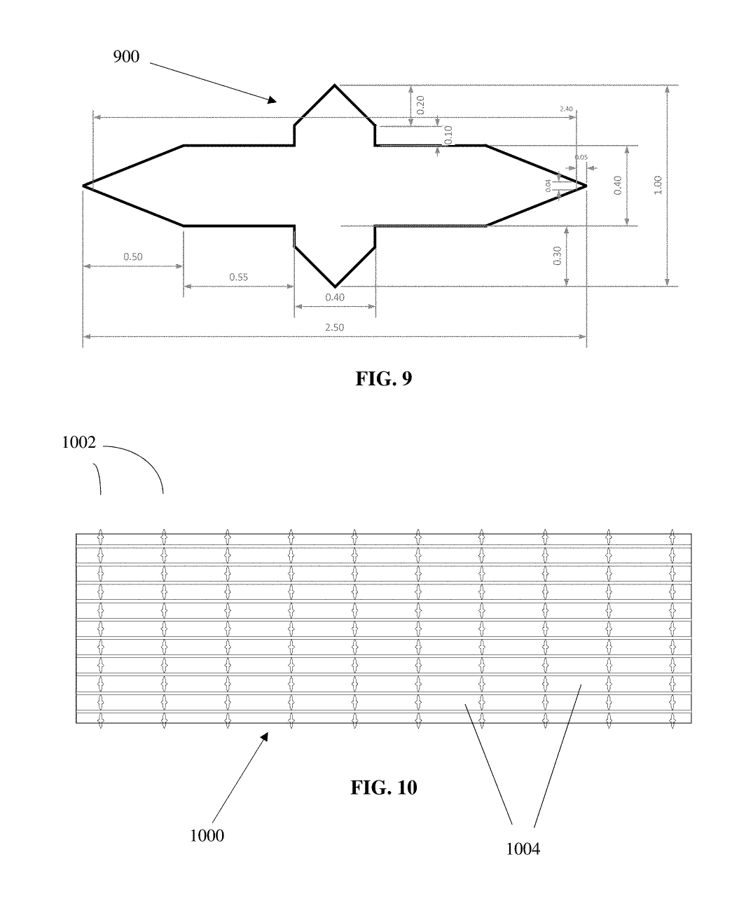

One current embodiment of a suitable emitter needle 900 is shaped like a cross as illustrated by FIG. 9. FIG. 9 provides dimension, are in mm, for a particular embodiment. Each emitter needle 900 resides in a purposefully formed receiving cavity provided by a reactor plate. The reactor plate also typically defines at least one, and typically plural, reaction microchannels.

For certain embodiments having multiple, simultaneously operating corona discharges, each reactor plate in a reactor can include plural electrodes, such as plural emitter needles. One embodiment of a reactor plate 1000 is illustrated by FIG. 10. Plate 1000 precisely spaces plural emitter electrodes 1002 and defines the reaction microchannels 1004 for conducting fluids, typically gases, used to perform chemical conversion reactions. FIG. 10 illustrates an embodiment having, for example, 10 corona discharge electrodes 1002 positioned in series along each of ten microchannels 1004. For one disclosed process embodiment, conversion of biogas to longer chain, typically liquid, hydrocarbons, it currently is assumed that an optimal process gas flow rate gas is 50 SCCM through a channel, such as microchannel 1004, having a square cross section with 0.4 mm edges.

Reactor tiles can be made of any suitable material, including metals and alloys, such as steel, aluminum, etc.; polymers, typically high temperature polymers; etc. Certain criteria that were applied to select preferred materials include: (1) the material is preferably easily formed, such as by injection molding or casting, although other processes, such as milling/machining stamping also may be used; and (2) the material should have good resistance to arcing. The best materials found to date for reactor tiles include phenolic resins, including glass-filled phenolic (“G-3”) resins, glass-filled silicone (“G-7”) resins, and castable ceramics, such as Portland Cement. For certain disclosed embodiments, the reactor tiles comprised Portland Cement and structural reactor features were formed in each tile by stamping.

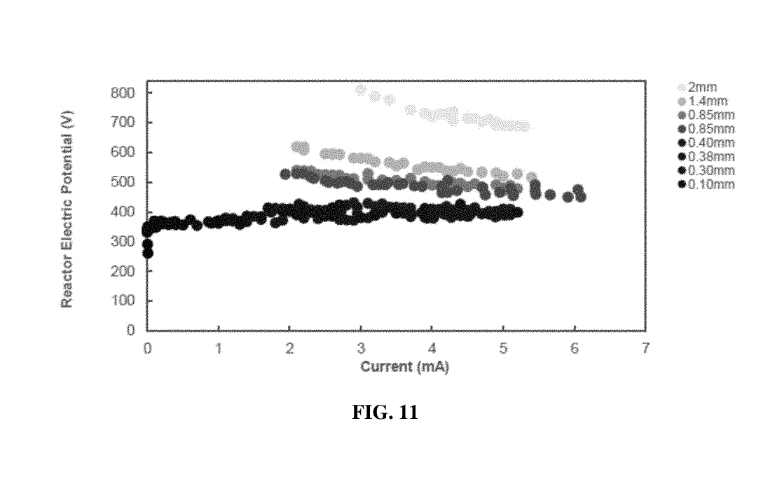

FIG. 11 is a graph of current (mA) versus reactor electrical potential for various different electrode placement distances. FIG. 11 illustrates determination of suitable placement distances for disclosed embodiments of the present invention.

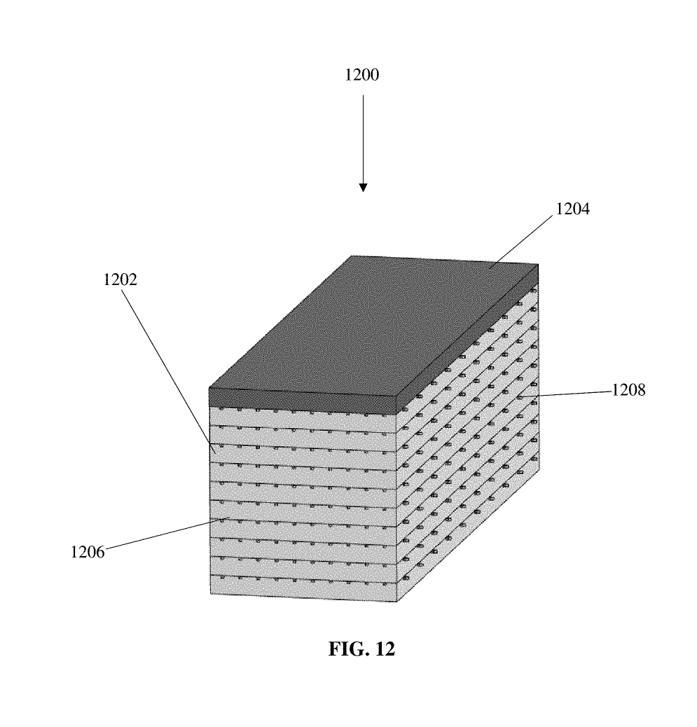

Individual reactor plates, such as plate 1000, may be assembled into a reactor stack. For example, and again with reference to an embodiment for converting biogas to longer chain, typically liquid, hydrocarbons, to enable a process inlet gas flow of 5 standard liters per minute (SLPM), a stack of 10 tiles per reactor assembly would be suitable. One embodiment of a suitable reactor stack 1200 is illustrated by FIG. 12. Stack 1200 comprises 10 reactor plates 1202 along with at least one outer sealing plate 1204 to seal the fluid channels. Each tile defines reactant inlet ports 1206 on the proximal face of reactor stack 1200, with product outlet ports (not shown) on the posterior face. Reactor stack 1200 includes a first set of electrodes 1208 shown on the right hand of the stack and a second set of electrodes (not shown) on the left hand side of the stack. A reactant distribution manifold (not shown) optionally may be operatively associated with the reactor stack to distribute reactants to the inlet ports 1206. For certain disclosed embodiments, the reactant pressure in will be from about 1 to about 10 bar (1 to 10 atmospheres; 14 psi to about 145 psi), and more typically from about 1 bar to about 5 bar (1 to 5 atmospheres; 14 psi to 73 psi) Likewise, a product collector distribution manifold optionally may be operatively associated with the product distribution end of the reactor stack 1200 to receive product and distribute such product to downstream components for further processing or product collection in a reservoir. A sealant and/or gasket can be used to provide a fluidly sealed device. One disclosed embodiment will use a spray silicone sealer (RTV like silicone) that will operate to both glue the plates together and seal any gaps between plates at minimal processing costs.

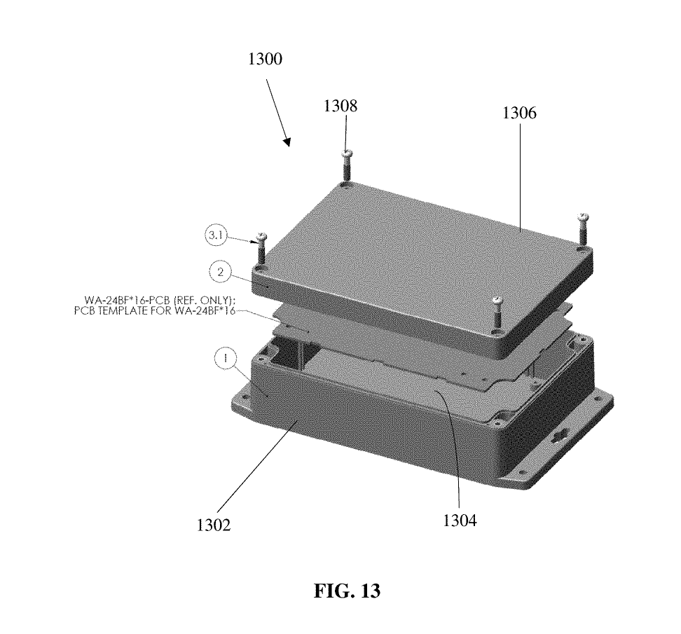

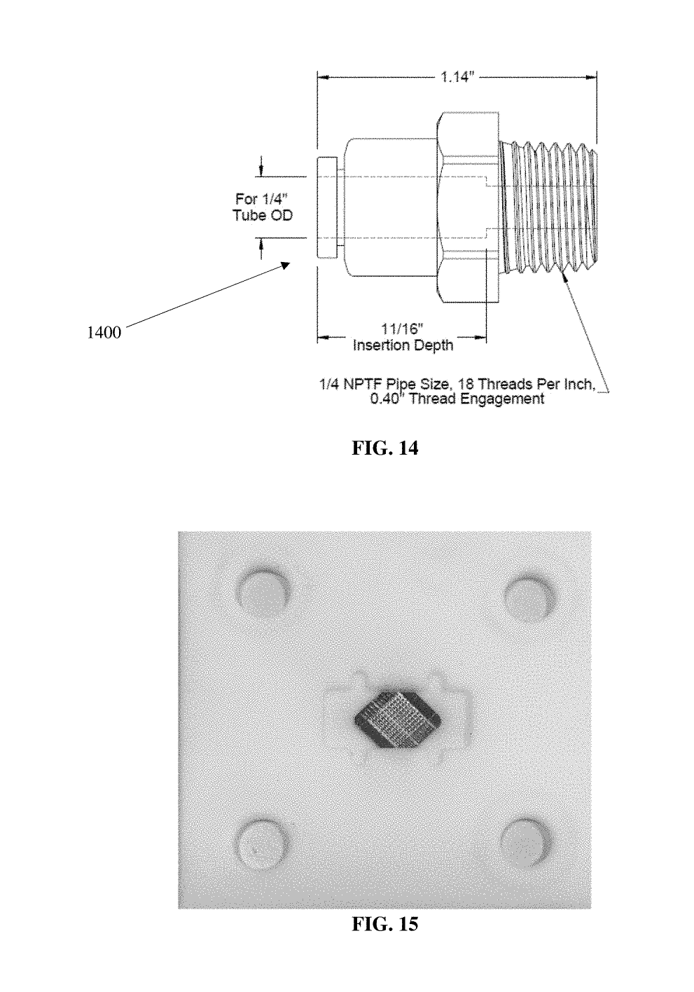

A reactor, plural separate reactors, a reactor stack or plural reactor stacks typically will be sealed in a housing. One embodiment of a reactor housing 1300 is illustrated by FIG. 13. Housing 1300 includes a bottom housing plate 1302 for housing at least one reactor or reactor stack 1304, a top housing plate 1306, a seal (not shown), and plural connectors 1308 for connecting the bottom and top housing plates to form the housing. FIG. 14 illustrates a case and push connector 1400 suitable for coupling external fluid flow lines to reactor housings such as housing 1300 according to the present invention.

For certain embodiments, an HVDC power supply powered the reactor. Certain disclosed embodiments need to operate up to 10×10×10 discharges in parallel, with each discharge consuming up to 5 W, more typically consuming up to 1 W (=1 kV×1 mA) electrical power. This equates to a power consumption of at least 1 kW for the reactor. Current embodiments use a boost converter design having suitably high power ratings to satisfy these and potentially higher power requirements.

In some embodiments, the DC power supply can be a sustained power supply. In some embodiments, the power supply can be sustained up to 10,000,000 Hz without any intrinsic frequency variations in voltage or current.

A reactor system according to the present invention can include a number of additional, potentially optional, components, as will be understood by a person of ordinary skill in the art. For example, a reactor system according to the present invention may include a pressure regulator, a backpressure regulator, a condenser, a liquid product collection reservoir, etc. Certain disclosed system embodiments included one, more than one in any and all combinations, or all of the following components: (a) microchannel reactor stack housing (e.g. modified Polycase WA-24); (b) Watts push to connect chemical resistant tubing connectors; (c) nylon tubing; (d) an inlet pressure regulator (e.g. a miniature plastic regulator capable of 20 SCFM); (e) an exit needle valve; (f) a heat exchanger (such as a fluid to air heat exchanger based on a simple coil of copper tubing); (g) a fluid, gas, liquid or both, collection tank [e.g. a 1,500 gal (64″×108″) tank is suitable for converting biogas to longer chain, typically liquid, hydrocarbons assuming 75% conversion to condensed liquids (biogas is 60% methane), and that 10 SCFH biogas will produce 0.65 gal liquids per day, where the tank can serve to accumulate products of several reactors].

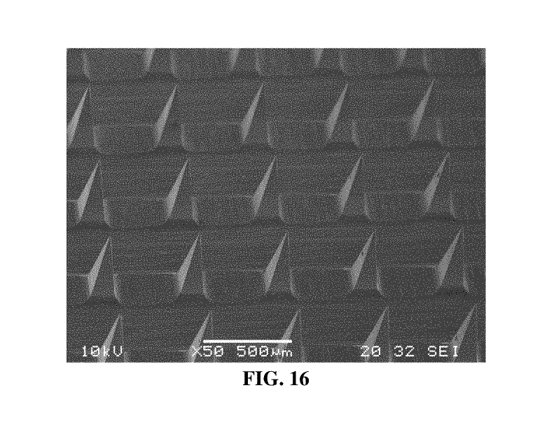

FIGS. 15 and 16 are images of a multi-discharge reactors according to the present invention. These FIGS. illustrate using plates having multiple emitter electrodes in an array.

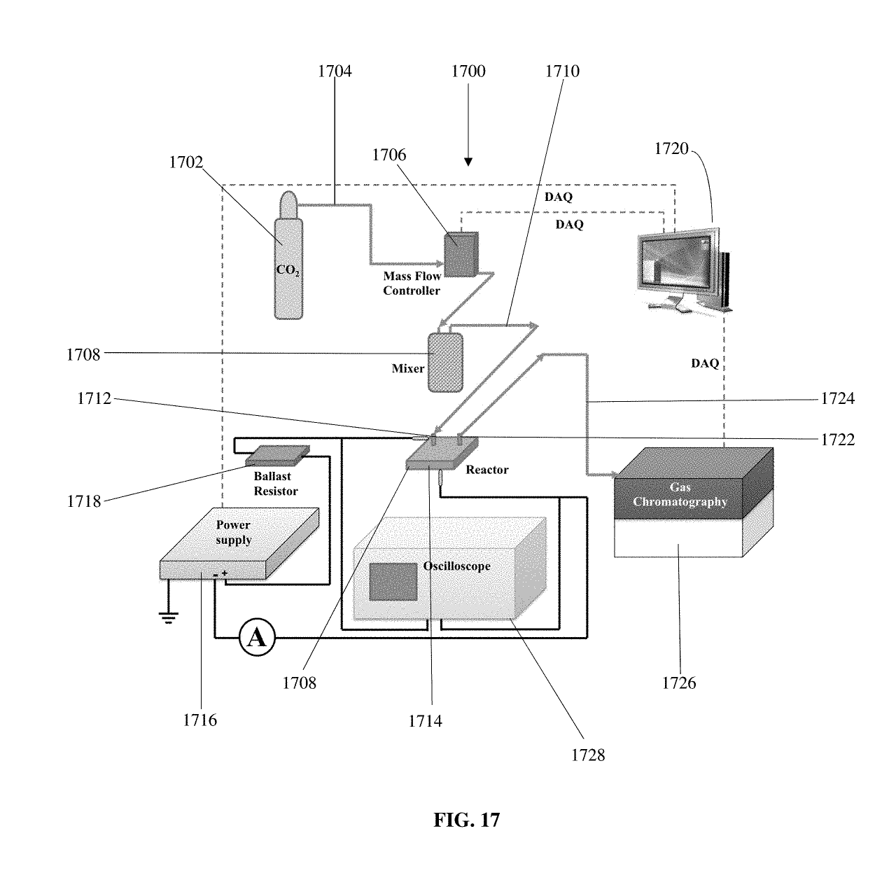

FIG. 17 illustrates a system 1700 that has been used to analyze products produced by subjecting a reactant stream comprising various gas compositions comprising CO₂ to corona discharge. System 1700 included a carbon dioxide source 1702 that was fluidly coupled via fluid line 1704 to a mass flow controller 1706 to precisely control the flow of CO₂ to a mixer 1708. Mixer 1708 formed various feed compositions comprising CO₂ for feeding via fluid line 1710 to a reactor inlet 1712 of corona discharge reactor 1714. Reactor 1714 was coupled to a power supply 1716 and a ballast resistor 1718. For this embodiment, system 1700 further included a computer 1720 for data acquisition. Reactor outlet port 1722 was fluidly coupled via fluid line 1724 to a gas chromatograph 1726 to analyze composition of products produced by reactor 1714. Finally, system 1700 also included an oscilloscope 1726 inline with the ballast resistor 1718 and reactor 1714.

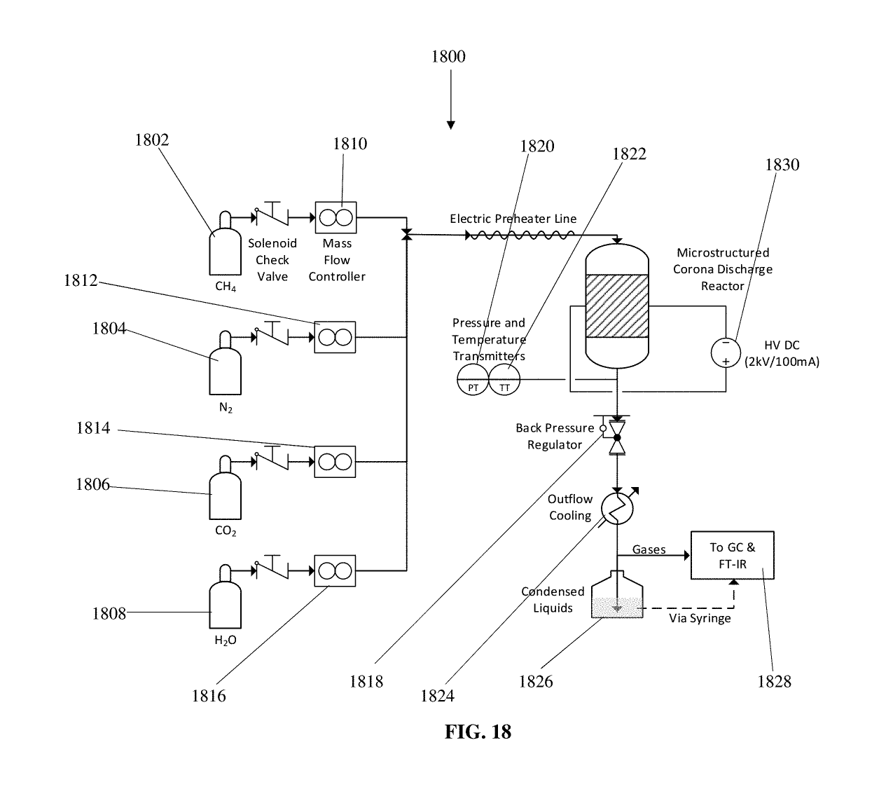

FIG. 18 illustrates an additional embodiment of a corona-discharge reactor system 1800 according to the present invention. FIG. 18 illustrates a Fluidic/Electric Balance of Plant (the F/E BOP) system 1800 that will be externally controllable using a Matlab/Simulink control panel. The F/E BOP 1800 includes plural gas reactant sources, such as methane (CH₄) source 1802; nitrogen (N₂) source 1804; carbon dioxide source 1806; and water (H₂O) source 1808. The F/E BOP may include mass flow controllers 1810, 1812, 1814 and 1816 associated with each of the plural gas reactant sources, thereby allowing a user to generate feed gas mixtures. System 1800 includes thermal and backpressure control subsystems, such as back pressure regulator 1816 to control physical reactor conditions. Pressure transmitters 1820 and temperature transmitters 1822 monitor reactor pressures and temperatures. Cooling device 1824, such as a heat exchanger, facilitates collecting product, such as condensed gas, in product reservoir 1826. System 1800 also may include instrumentation to facilitate product composition analysis, such as on-line gas chromatograph and/or spectrometers, such as an online FT-IR with ATR attachment and an online Raman gas analyzer, collectively shown generically as 1828 in FIG. 18. System 1800 also includes a power supply 1830 capable of fast transition from high voltage/low current (˜2 kV/1 nA) to lower voltage/higher current (˜300V/100 mA).

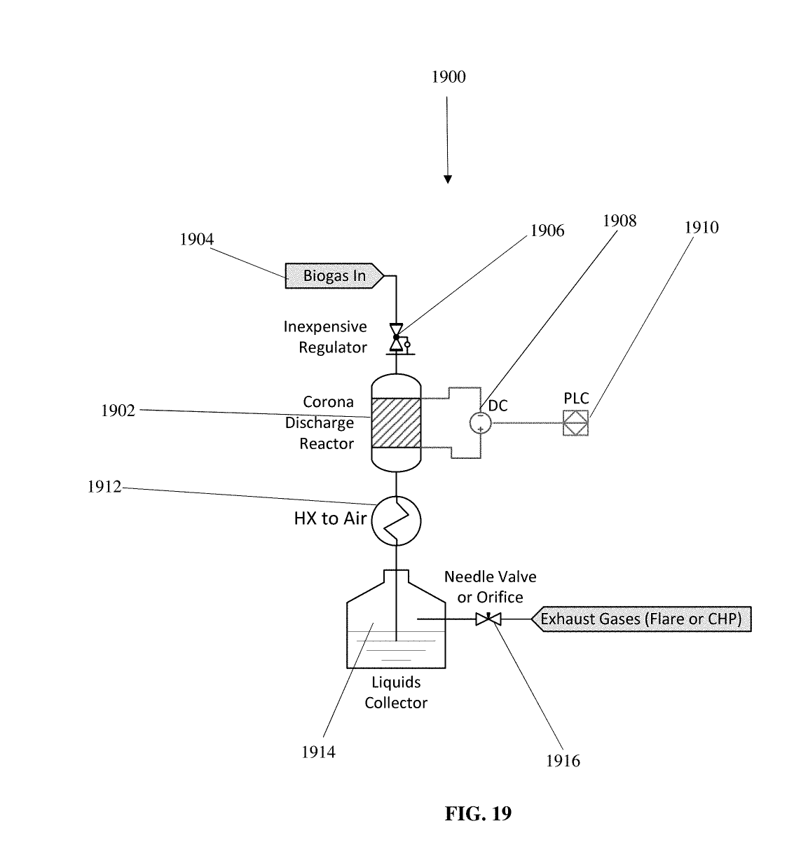

FIG. 19 illustrates one embodiment of a commercial system 1900 useful for converting biogas to higher condensed hydrocarbons. System 1900 comprises a corona discharge reactor 1902 according to the present invention configured for converting biogas to higher condensed hydrocarbons. System 1900 components include a source of biogas 1904; a pressure regulator 1906 to control reactor inlet pressure; corona discharge reactor 1902 coupled to DC power source 1908 that is controlled by power supply control unit 1910; a heat exchanger 1912, such as an air-cooled heat exchanger, to facilitate recovering condensable products generated by the reactor as liquids; a liquids collection reservoir 1914; and a back pressure regulator 1916 to control reactor exit pressure. System components may be shared between reactors. For example, a liquid products reservoir can be shared between many modules, whereas an exit pressure regulator preferably is dedicated to each module. Likewise, it is likely that one HV power supply is sufficient to support many reactor modules using an appropriate power distribution process.

Certain embodiments of disclosed corona discharge microreactors are designed to increase flexibility. For example, such embodiments may include adjustable corona discharge gap sizes, a view port to view the discharge, ports/appropriate connections to allow spectroscopic measurements of the discharge to characterize different operational modes. Other embodiments will be designed to include predetermined, selected features, such as microchannel size (cross section in width and depth, as well as spacing between discharges), number of active discharges, etc.

FIG. 34 illustrates an embodiment of a microreactor 3400 comprising a plate counter electrode 3402, a spacer 3404, and a reactor plate 3406 comprising channels 3408.

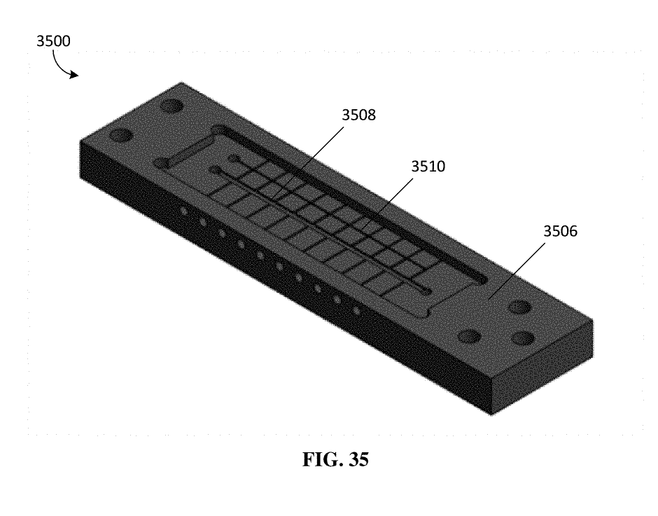

Certain disclosed embodiments may include an inert gas inlet and microchannel to provide inert gas around and about the needle tip emitter electrodes. For example, FIG. 35 illustrates a portion of a micro-reactor 3500 comprising reactor plate 3506, channels 3508, and an inert gas channel, such as channel 3510, to feed an inert gas around the needle tip emitter electrodes. The immediate environment surrounding the needle tips can be bathed with inert gas, thus allowing the electrons to lower their overly high energy (e.g., thermalize), overly high electron energy can cause carbon deposition on the needles.

The inert gas channel can be located within the reactor geometry and can be configured to convey inert gas to the emitter electrodes. The inert gas channel can serve each electrode separately and can change the reaction environment around each electrode. This allows for a distributed, near continuous feeding of reactants along the length of the reactor.

In some embodiments, the reactant gas can flow perpendicularly to the electrodes and the plasma region. In other embodiments, such as embodiments having an inert gas channel, the reactant gas may flow in a non-perpendicular direction. For example, the gas may enter along the emitter needle electrodes and into the corona space.

In some embodiments, the microreactor can be configured to function without a dielectric protection coating anywhere in the reactor. This configuration can greatly increase the energy efficiency of the reactor. Additionally, the lack of dielectric material makes it possible, in some embodiments, to use AC current in an energy efficient mode without overheating.

In some embodiments, one or more corona discharge reactors can be integrally combined with one or more additional and separate units, such as a catalytic reactor. Integrating a catalytic reactor with a corona reactor allows specific molecules that are obtained in the corona reactor to be further processed by the catalytic reactor. This combination eliminates the need to use a separation unit to first separate molecules obtained in the corona reactor. The integrated catalytic reactor can transform the available molecules selectively, for example, by taking from the mixture only those molecules that can be catalytically transformed.

### IV. Operating Characteristics of Disclosed Corona Discharge Reactors

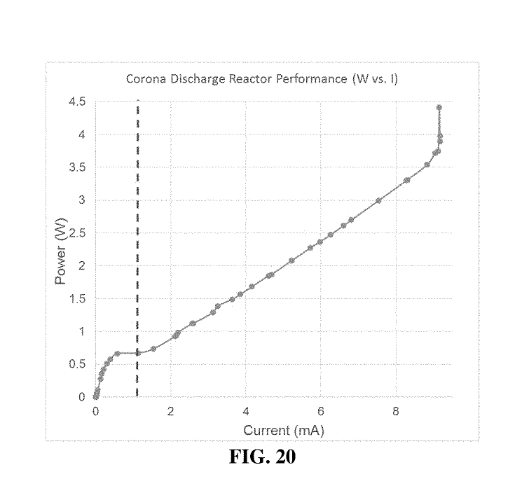

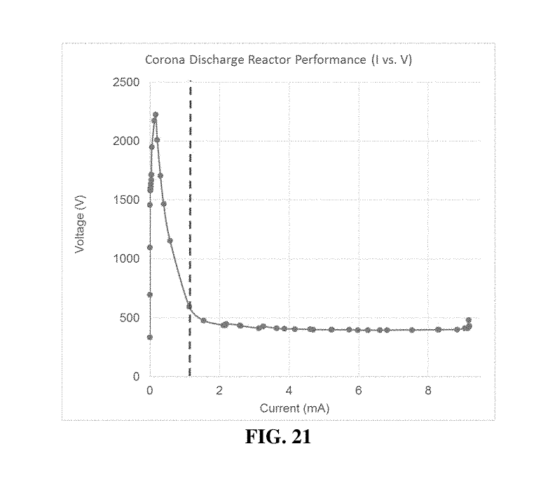

Disclosed systems can create a stable glow corona, with the process being controlled by total power applied to the reactor at a given current, and allowing the potential (voltage) to be as high as needed. The electrical performance of an exemplary system is shown in FIG. 20 and FIG. 21, with FIG. 20 showing the applied power as a function of current, and FIG. 21 showing the resulting voltage profile needed to allow current flow. FIG. 20 illustrates that controlling total power into the system controls the discharge output. The total power into the system should be, for certain disclosed embodiments, between about 1 to about 10 mA, and more typically between about 1.5 mA to about 9 mA. FIGS. 20 and 21 illustrate that after a threshold potential is exceeded, and if a more than a specific total power is applied, a stable electron cascade creates a column of electrons flowing through a gas and creates a closed electric circuit, which is referred to herein as a stable glow discharge. For example, with reference to FIG. 20, after about 0.7 W was applied to the gap, fluid in the gap became sufficiently ionized to be conductive and stabilized the corona discharge. This threshold is indicated by the red vertical dashed line in FIG. 21. FIG. 21 shows that initially there is a large increase in voltage due to electrical breakdown of any gas in the gap between the two electrodes. A person of ordinary skill in the art will appreciate that the gas in the gap can have varied compositions, and likely is determined by appropriate selection of an inlet reactant gas. The voltage required to initially induce gas breakdown can vary as will be understood by a person of ordinary skill in the art, as is described by Paschen's law. Applied voltage needs to be controlled too to produce a corona discharge, an arc discharge, or a pulse discharge, as desired, most typically a corona discharge. With reference to FIG. 21, the corona discharge region is that region at about 500 volts and at a current greater than that represented by the dashed line. One aspect of the present invention is recognizing the benefits associated with operating disclosed embodiments to proceed smoothly down the backside of the voltage spike at about 500 volts to place the device in a corona discharge regime.

Under stable glow discharge operation, high energy free electrons in the glow discharge collide with molecules in the gas, yielding reactive species, including ions and free radicals. When operating using methane as the reactant gas, for example, it appears that a significant number of radicals (e.g. CH₃. and CH₂: radicals) form in the gas phase that can dimerize to form ethane and ethylene. This happens with high efficiency, with methane conversion rates growing as a function of applied power (essentially, increased current at constant voltage).

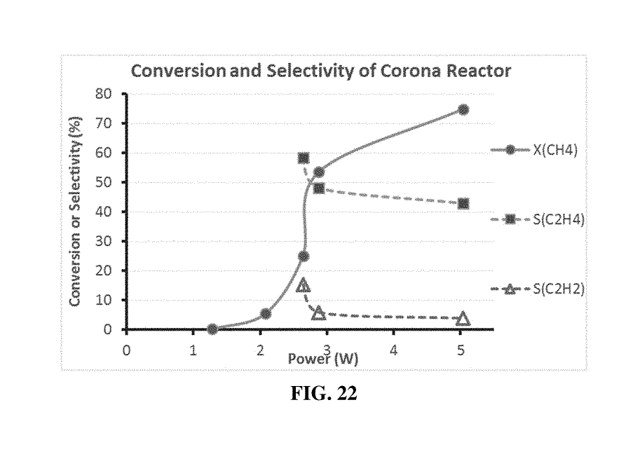

Data collected using a single discharge reactor performing chemical condensation of methane is shown in FIG. 22. FIG. 22 shows initial low conversion at low power, since this takes place in a spark discharge regime characterized by the very short duration emission of a few extremely energetic electrons. As a consequence, only a few CH₄ molecules can be activated, and most of the energy of the electrons is wasted. As power increases a stable glow discharge is established, characterized by higher currents (i.e., higher electron flux) at lower voltages (i.e., less energy per electron, resulting in more thorough use of the energy of each electron). As such, many more CH₄ molecules are activated, leading to higher conversion with higher overall energy efficiency. Eventually, a saturation level is encountered, where increases in power do not lead to increased methane conversion because an overabundance of electrons causes many electrons to be wasted, or to collision with previously activated gas molecules (resulting in the formation of carbon black). A lower density of free electrons therefore leads to more efficient utilization of to drive chemistry. It appears that after a certain current density, free electrons start encountering molecules that have already been activated towards reaction, leading to unproductive collisions, and wasted energy.

This saturation behavior is reflected in the data in FIG. 22, and can be compared with the data disclosed by FIG. 4 of “Direct Conversion of Methane and Carbon Dioxide to Higher Hydrocarbons Using Catalytic Dielectric-Barrier Discharges with Zeolites” B. Eliasson, C.-J. Liu, U. Kogelschatz, Ind. Eng. Chem. Res. 39, 1221-1227 (2000). The saturation observed as more power is delivered after about a delivery of 500 W to the reactor disclosed by Eliasson et al. indicates that the marginal efficiency of each additional unit of energy delivered to the gas is significantly less productive after a certain level. FIG. 22 illustrates that, at initiation, very little reactant conversion to product occurs, as the discharge reactor operates in spark discharge mode. As power input increases, the reactor rapidly converts from a spark discharge mode to a corona discharge mode and conversion of reactant to product increases from virtually 0% to about 60%.

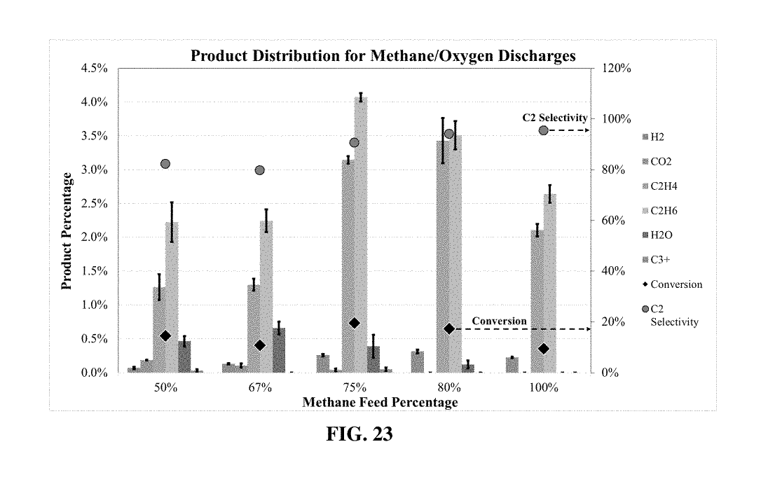

The discharge test loop of FIG. 1 was used to perform coupling reactions, such as methane coupling, by applying corona discharge to mixtures of CH₄ and O₂. FIG. 23 shows product distributions, conversion, and selectivity to C₂ hydrocarbons results for a single pass with a residence time of 0.4 ms. The discharge is sustained by 800 V or less at the reactor with a current of 1.8 mA, resulting in power in the range of 1.5 watts. At a CH₄:O₂ ratio of 3:1, methane conversion is just under 20% and the selectivity towards C₂ products is ˜90%. Eliasson et al. discloses conducting a reaction by delivering 500 W to a flow of 150 sccm (3.33 J/ml). In striking contrast, the data provided by FIG. 23 are the result of applying 1.5 W to a flow of 20 sccm methane (˜0.1 J/ml!).

Newly created C₂ molecules can be exposed to further electron cascades, causing, for example, ethylene to react with methylene to produce C3 compounds, or ethylene radicals to form C4 and heavier hydrocarbons. This process allows methane to be converted to longer chain hydrocarbon liquids with high efficiency. The composition of the resulting product streams can be appropriately characterized, such as spectroscopically, and/or gas chromatography.

One embodiment of a corona discharge reactor platform comprises arrays of emitter electrodes to drive sequential conversion reactions. One embodiment of a disclosed reactor will operate on flows in the order of 600 SCCM with 80% selectivity conversion rates towards condensed hydrocarbons. The estimated pressure drop is about 0.2 bar using the Churchill capillary flow correlation for a 100 sccm flow of air at 100° C. and pressure 2.5 bar through a rectangular capillary of dimensions 0.2×1.0×90 mm³ with a roughness of about 0.001 m. This estimated pressure drop indicates that a flow rate of 100 sccm of natural gas through this embodiment of a reactor is reasonable.

### V. Method of Using a Corona Discharge Reactor/System Comprising a Reactor

A. Chemical Transformations

A corona discharge microreactor, an array of microreactors, a system comprising at least one microreactor, or a system comprising an array of microreactors can be used to convert a feed stream, typically a feed gas, into a desired product or products. A person of ordinary skill in the art will appreciate that any reaction that is facilitated by or induced by energy transfer from a corona discharge, such as a gas phase reaction, and in particular gas phase reactions that proceed by radical formation, can be accomplished using disclosed embodiments of the corona discharge device or system comprising the device.

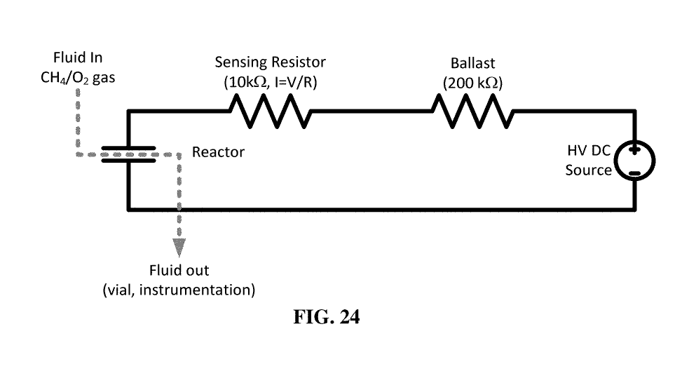

One particular embodiment of the present invention concerns using corona discharge for highly efficient conversion of a low molecular weight hydrocarbon, such as methane (CH₄) (e.g., from natural gas, or from anaerobic digestion of waste biomass) to carbon-based materials having a larger number of carbon atoms in a chain, such as hydrocarbons having from 2 to about 100 carbons (C₂-C₂₀) in a chain, more typically from 4 to about 20 carbon atoms (C₄-C₂₀) in a chain, and even more typically liquid transportation fuels in the gasoline (hydrocarbons of about C₁₀) or diesel (hydrocarbons of about C₁₆) range. Corona discharge has been used convert methane streams to C₂ and C₃ molecules, such as ethane, ethylene, acetylene, propane, propene, etc. A general schematic of one working embodiment of the process is illustrated in FIG. 24.

Without being bound by a theory of operation, it currently is believed that the corona discharge methane condensation process occurs in several steps. In a first step that has been established empirically, methane (CH₄) is activated by a highly energetic electron represented as [symbol] , in Equation (1) (the electron is not consumed in the reaction, but instead flows to a counter electrode).

2CH₄+ [symbol] →C₂H₆+H₂ Equation (1)

If a second discharge is implemented in tandem with a first discharge then additional reactions are possible, as indicated by equations 2 and 3.

C₂H₆+CH₄+ [symbol] →C₃H₈+H₂ Equation (2)

2C₂H₆+ [symbol] →C₄H₁₀+H₂ Equation (3)

Based on working example experience, approximately 70% methane conversion occurs in the first discharge with the major products being ethane, ethylene and acetylene. Likely products for the second discharge will be those described by Equations 2 and 3. Eventually, and following a number of sequential corona discharges, a product of a certain desired length n will be formed in an overall reaction that can be summarized as

nCH₄+n/2 [symbol] →CₙH₂ₙ₊₂+nH₂ Equation (4),

where n is from 2 to at least as high as 20, more typically 4 to 18, such as 4 to 16 or 4 to 10 or 12. A “longer” (longer than the starting material) chain hydrocarbon product is then captured and removed from the reactor. In order to achieve high energy efficiency, hydrogen (H₂) produced by the reaction is preferably used for a constructive purpose (e.g., in a fuel cell to produce electrical power that can be used to run the process). Chain length control approaches include decreasing vapor pressure of hydrocarbons as their chain lengths increase; controlling reactor temperature; condensing hydrocarbons out having a targeted, or longer, chain length; and combinations thereof. Disclosed embodiments can convert a reactant stream to liquid hydrocarbon product wherein 95% of the liquid hydrocarbon produced is between n+2 and n−2 of the target n set by external process control. A similar transformation (i.e., production of long chain hydrocarbons from methane) is currently accomplished by reforming methane to syngas (an energy intensive process) followed by Fischer-Tropsch synthesis (FTS) to liquids. The present process is superior to the current implementation of steam methane reformation followed by Fischer-Tropsch synthesis as production of waxes and of very small hydrocarbons is completely avoided. And, the overall energetic efficiency of a combined reforming/FTS process is about 50%. The energy efficiency of the corona discharge should be significantly higher, such as about 75% or better.

B. Fluid Purification

Corona discharge reactors, or a system comprising at least one corona discharge reactor, also can be used for fluid purification. One important exemplary embodiment of fluid purification is water purification. Potable water to substantially pure water can be produced from impure water by applying to the impure water an electron stream from a corona discharge reactor. This embodiment has been modeled using an input composition comprising water and rhodamine red. Rhodamine red was substantially eliminated as the contaminant from water by treating the water with a disclosed embodiment of a corona discharge reactor. For this embodiment, the reactor process conditions were: voltage=500V; current=5 mA; flowrate=2 sccm. The discharge occurred in the headspace gas above the liquid, with discharge occurring from the emitter to the liquid (liquid=anode).

A second exemplary embodiment of a fluid purification process is desulfurization of a hydrocarbon stream. Hydrocarbon fuels are often contaminated with sulfur-based compounds, such as thiophenes, including dibenzothiophenes and substituted dibenzothiophenes. These compounds are difficult to remove using prior known processes. However, a product stream comprising a hydrocarbon and a sulfur-based impurity can be exposed to a corona discharge. For example, a hydrocarbon comprising a thiophene can be oxidatively desulfurized by converting the thiophene to a sulfone and/or sulfoxide, as indicated below, using corona discharge.

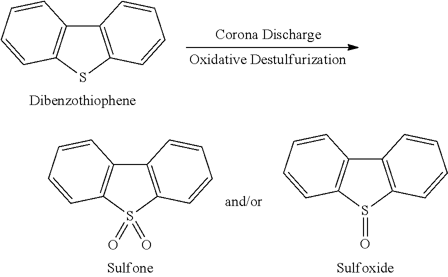 [SMILES: c1ccc2c(c1)sc1ccccc12 ; O=S1(=O)c2ccccc2-c2ccccc21 ; O=S1c2ccccc2-c2ccccc21]

The sulfone or sulfoxide is then removed from the hydrocarbon stream by liquid-liquid extraction to remove the sulfur-based impurity(ies).

Natural gas may comprise components such as CO₂, water and H₂S. Corona discharge has been applied to mixtures of CH₄, CO₂, and H₂O. Methane (CH₄) has been produced from a mixture comprising CO₂ and H₂O, establishing that neither of these two gases will negatively impact the process. Further, corona discharge effectively precipitates sulfur out of CH₄/H₂S mixtures. This allows both a method for purifying a fluid stream comprising H₂S using corona discharge, and also establishes that an inlet feed stream comprising H₂S can be processed using corona discharge to produce hydrocarbons.

C. Forming Reactive Nitrogen Products

Another example of a useful process that can be accomplished using a corona discharge reactor is conversion of nitrogen to a reactive nitrogen form, such as ammonia or hydrazine. This has been accomplished by, for example, applying a corona discharge to a mixture of nitrogen and oxygen, to yield nitrogen oxides that can be further processed to nitrates, or to a mixture of nitrogen and water vapor, to yield ammonia and hydrazines. The process parameters were substantially similar to those described above for gas phase processes, with voltages of about 600V and current of about 5 mA applied to a microchannel reactor with a gap of about 1 mm.

D. Carbon Dioxide Reduction

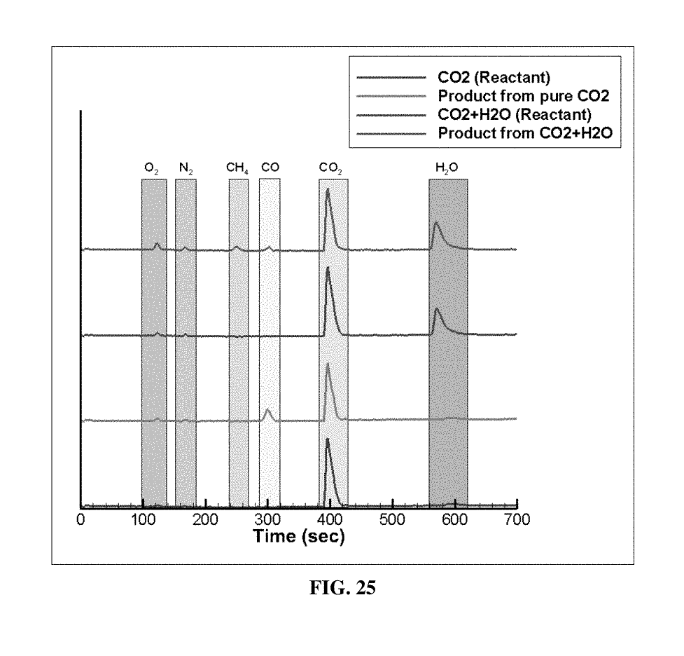

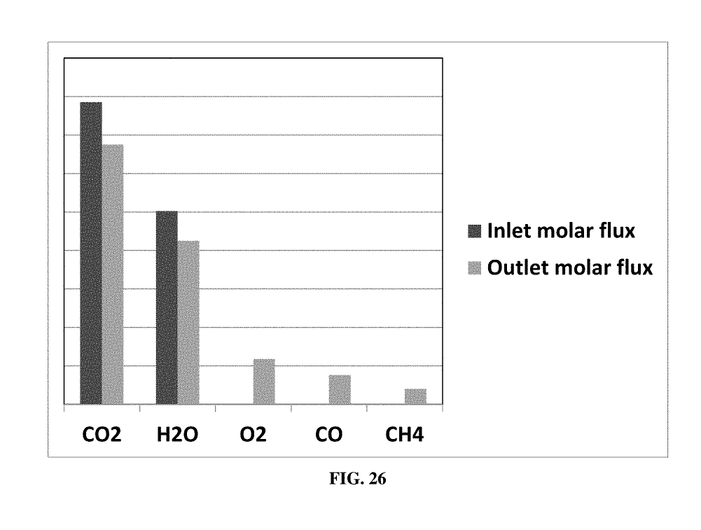

Another disclosed process concerns CO₂ reduction. FIG. 17 illustrates a system 1700 that has been used to analyze products produced by subjecting a reactant stream comprising various gas compositions comprising CO₂ to corona discharge. FIGS. 25 and 26 provide the gas chromatography product analysis results of products produced using the system according to FIG. 17. FIGS. 25 and 26 establish that reactant compositions comprising CO₂, including compositions comprising CO₂ and H₂O, subjected to corona discharge produce, for example, oxygen (O₂), nitrogen (N₂), methane (CH₄), carbon monoxide and (CO).

### VI. Examples

The following examples are provided to illustrate certain features of exemplary embodiments. A person of ordinary skill in the art will appreciate that the scope of the present invention is not limited to features disclosed by such examples.

### Example 1

A typical 1,000-cow dairy is assumed to produce 50 ft³/cow/day biogas comprising about 60-70% methane and 30-40% CO₂. The inlet pressure for this biogas to a corona discharge reactor would be about 1.5 bar. Thus, the dairy will produce 30 ft³/cow/day or 30,000 ft/day methane (equivalent to 1,250 ft³/hour). Since the current major use of biogas (where it is used constructively) is for production of electrical power through use of electrical generators coupled with a diesel-generator (genset), the present evaluation will be compared with the use of a genset (as far as capital costs and value of products generated).

### Example 2

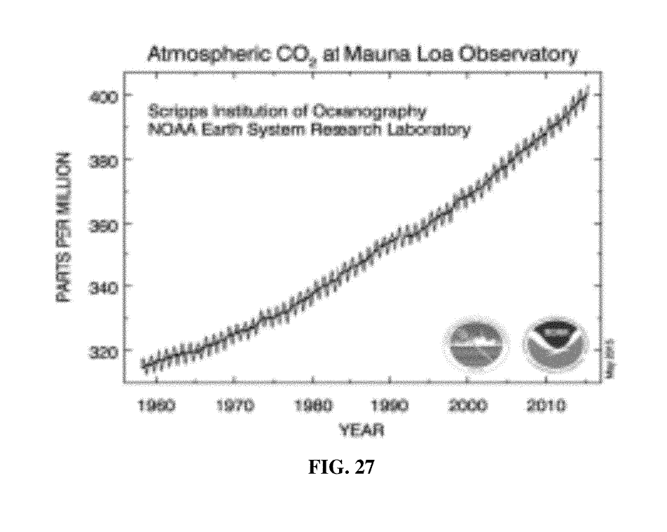

This example concerns using a corona reactor to reduce CO₂ in a fluid inlet stream using a microreactor. Atmospheric CO₂ has increased substantially in the last 50+ years. Annual measurements of atmospheric CO₂ in parts per million are recorded at Mauna Loa Observatory. FIG. 27 is a graph of year versus atmospheric CO₂ concentration in parts per million from 1958 to 2015. Embodiments of the present invention can be used to reduce CO₂. Using a system according to FIG. 17, fluid reactant streams comprising CO₂ were subjected to corona discharge. The results are provided by FIGS. 25 and 26.

### Example 3

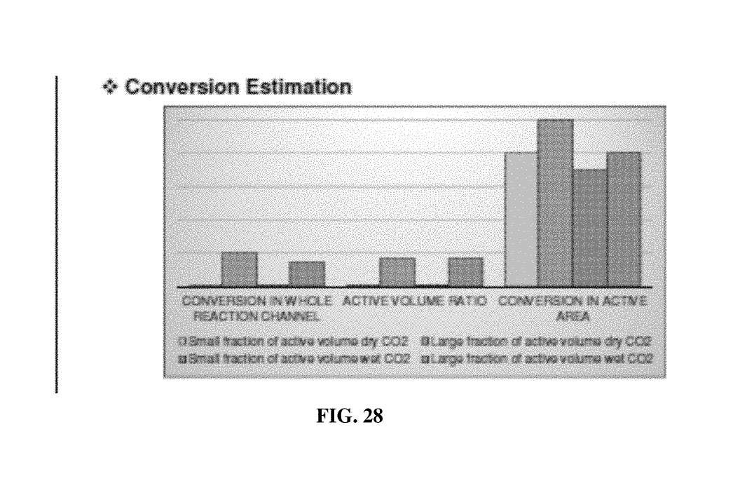

This example concerns using a three discharge corona reactor to reduce CO₂ in a fluid inlet stream using a microreactor. A reactor such as that illustrated by FIG. 6 was used to subject fluid streams comprising CO₂ to plural corona discharges. Increasing the fraction of active volume increased the conversion of CO₂ in the whole channel and improved the reactor efficiency. These results are provided by FIG. 28.

### Example 4

This example concerns using a corona reactor to reduce CO₂ in a fluid inlet stream using a microreactor. One embodiment of a multi-discharge reactor used for this conversion is illustrated in FIGS. 15 and 16.

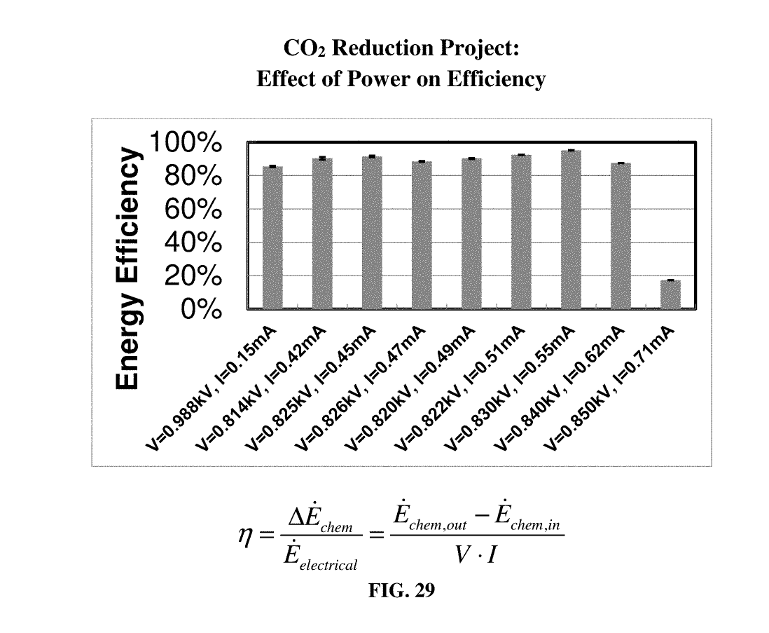

Data concerning the effect of power on efficiency for CO₂ reduction using a corona discharge reactor according to the present invention is provided by the bar graph of FIG. 29.

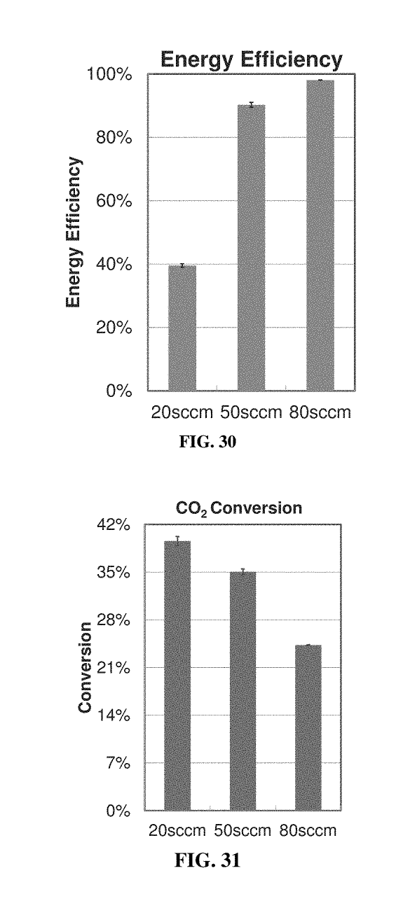

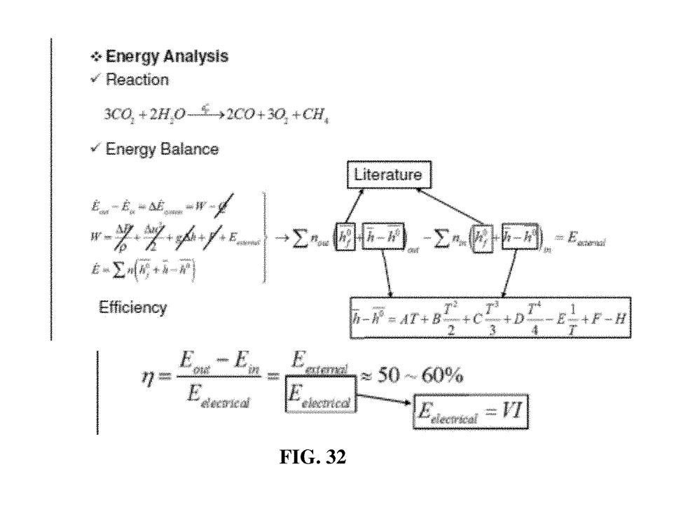

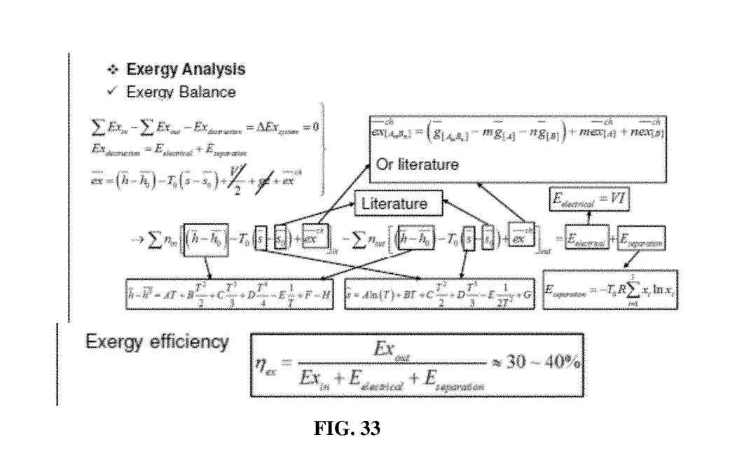

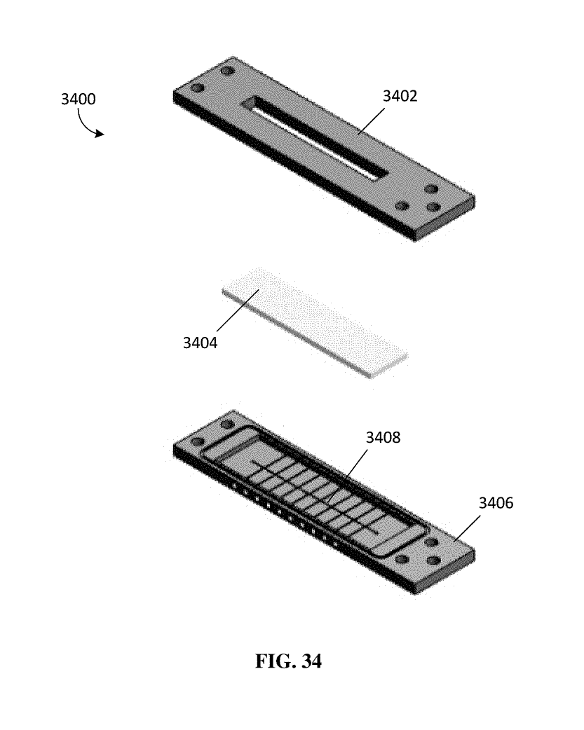

FIG. 30 is a bar graph illustrating energy efficiency for CO₂ reduction using a corona discharge reactor according to the present invention at various different feed (20, 50 and 50 standard cubic centimeters per minute (sccm)) rates.

And FIG. 31 is a bar graph illustrating percent conversion to products for CO₂ reduction using a corona discharge reactor according to the present invention at various different feed (20, 50 and 50 standard cubic centimeters per minute (sccm)) rates.

In view of the many possible embodiments to which the principles of the disclosed invention may be applied, it should be recognized that the illustrated embodiments are only preferred examples of the invention and should not be taken as limiting the scope of the invention. Rather, the scope of the invention is defined by the following claims. We therefore claim as our invention all that comes within the scope and spirit of these claims.

We claim:

1. A microreactor configured to produce a corona discharge.

2. The microreactor of claim 1, further comprising:
one or more emitter electrodes and one or more counter electrodes having a fluid channel therebetween, wherein the channel is configured such that one or more reactants flowing through the channel flow perpendicularly with respect to the one or more emitter electrodes and the one or more counter electrodes, the emitter and counter electrodes configured to produce plural, simultaneously operating corona discharges; and
a DC power source coupled to the reactor and controlled by a power source control unit.

3. The microreactor of claim 2, wherein the emitter electrodes are needle tip electrodes and the counter electrodes are plate electrodes, and wherein the electrodes comprise a material selected from a group consisting of a metal, a metal alloy, a super alloy, a semiconductor, or combinations thereof.

4. The microreactor of claim 2, wherein the channel has a width of no more than 500 μm.

5. The microreactor of claim 2, wherein the DC power source comprises one or more in-line ballast resistors.

6. The microreactor of claim 2, configured to produce 2 to 100 simultaneously operating corona discharges.

7. The microreactor of claim 2, wherein the emitter electrodes are cross-shaped.

8. The microreactor of claim 2, further comprising a reactor plate configured to receive and position the one or more emitter electrodes.

9. The microreactor of claim 1, configured to produce multiple simultaneously operating corona discharges.

10. The microreactor of claim 1, wherein the corona discharge is a non-thermal corona discharge having a temperature not exceeding 100° C.

11. A microreactor system, comprising:
a plurality of microreactors assembled into a reactor stack, each microreactor comprising: a reactor plate, one or more emitter electrodes and one or more counter electrodes configured to provide plural, simultaneously operable corona discharges, the emitter electrodes and the counter electrodes having a fluid channel therebetween such that one or more reactants can flow through, the fluid channel comprising a reactant inlet port and a product outlet port;
a reactant distribution manifold coupled to the reactant inlet ports;
a product collection manifold coupled to the product outlet ports and configured to receive product produced by the reactor stack; and
a DC power supply coupled to the electrodes.

12. The microreactor system of claim 11, further comprising at least one of a computer configured to control at least one of operating parameters and data acquisition, product analytic instrumentation, a pressure regulator, a condenser, a heat exchanger, a pressure transmitter, a temperature transmitter, and analytic instrumentation.

13. The microreactor system of claim 12, wherein the analytic instrumentation comprises at least one of a gas chromatograph, a mass spectrometer, FT-IR spectroscopy, a Raman gas analyzer, or combinations thereof.

14. The microreactor system of claim 11, wherein the DC power supply is configured for transition from high voltage/low current (˜2 kV/1 nA) to lower voltage/higher current (˜300V/100 mA).

15. The microreactor system of claim 11, wherein the one or more reactants are selected from a group consisting of methane (CH₄), nitrogen (N₂), carbon dioxide (CO₂), or water (H₂O).

16. The microreactor system of claim 11, further comprising a mixer coupled to the reactant distribution manifold, the mixer configured to receive plural reactants and to form mixtures thereof.

17. A method for using a microreactor according to claim 1, or a microreactor system according to claim 11, comprising:
generating one or more simultaneously active corona discharges;
performing at least one of chemical transformation and product purification on a reactant stream.

18. The method of claim 17, wherein the act of performing a chemical transformation on a reactant stream comprises condensing a reactant stream comprising methane to produce C₂ or greater hydrocarbons.

19. The method of claim 17, wherein the reactant stream is biogas from a dairy.
